(19) United States
(12) Patent Application Publication
(10) Pub. No.: US 2016/0287549 A1
(43) Pub. Date: Oct. 6, 2016

(54) NOVEL METHODS FOR TREATING NEURODEGENERATIVE DISEASES

(71) Applicant: GENZYME CORPORATION, Cambridge, MA (US)

(72) Inventor: James DODGE, Shrewsbury, MA (US)

(21) Appl. No.: 15/038,185

(22) PCT Filed: Nov. 21, 2014
(86) PCT No.: PCT/US2014/066766

§ 371 Date: May 20, 2016

Related U.S. Application Data
(60) Provisional application No. 61/908,019, filed on Nov. 22, 2013.

(51) Int. Cl. A61K 31/277

(52) U.S. Cl. CPC A61K 31/277

(57) ABSTRACT

The invention relates to dihydroorate dehydrogenase (DHODH) inhibitors useful for the treatment of neurodegenerative diseases, such as amyotrophic lateral sclerosis.

### CROSS-REFERENCE TO RELATED APPLICATION

This application claims the benefit of priority of U.S. Provisional Patent Application No. 61/908,019, filed Nov. 22, 2013, which is hereby incorporated by reference in its entirety.

### FIELD OF THE INVENTION

The present invention relates to the field of therapeutics for neurodegenerative diseases. More specifically, the invention relates to dihydroorate dehydrogenase (DHODH) inhibitors useful for the treatment of neurodegenerative diseases, such as amyotrophic lateral sclerosis.

Amyotrophic lateral sclerosis (ALS) is a progressive, neurodegenerative condition involving the loss of large motor neurons in the brain and spinal cord. It is characterized by progressive weakness, atrophy and spasticity, leading to paralysis and respiratory failure within five years of onset. Familial ALS accounts for 10% of all ALS cases; approximately 25% of these cases are due to mutations in the Cu/Zn superoxide dismutase gene (SOD1). To date over 10 different mutations have been identified in the SOD1 gene spanning all five exons. SOD1 is a mainly cytoplasmic enzyme that catalyzes the breakdown of superoxide ions to oxygen and hydrogen peroxide, which in turn is degraded by glutathione peroxidase or catalase to form water. Several lines of evidence indicate that mutant SOD1 protein is neurotoxic through an acquired, adverse function that entails both oxidative pathology and protein aggregation, with secondary disturbances of glutamate metabolism, mitochondrial function, axonal transport and calcium homeostasis. That mutant SOD1 is toxic is strongly supported by the observation that transgenic expression of high levels of mutant SOD1 protein in mice produces a motor neuron disease phenotype, with age of onset and disease duration dependent on copy number.

To date, few therapeutic interventions have altered the motor neuron phenotype in the transgenic ALS mice. Accordingly, there is a need for developing novel therapies for treating neurodegenerative diseases, such as ALS.

### SUMMARY OF THE INVENTION

The present invention is based, at least in part, on the discovery that inhibitors of dihydroorotate dehydrogenase (DHODH) are effective in treating neurodegenerative diseases such as ALS. The invention features methods for using DHODH inhibitors to treat a subject having or at risk of having a neurodegenerative disease (e.g., ALS). The invention also features compositions for use in treating a subject having or at risk of having a neurodegenerative disease (e.g., ALS).

In aspects, the invention provides methods for treating ALS in a subject. In embodiments, the methods involve administering a therapeutically effective amount of a dihydroorate dehydrogenase (DHODH) inhibitor to the subject.

In aspects, the invention provides methods for delaying mortality in a subject having or is at risk of having amyotrophic lateral sclerosis (ALS). In embodiments, the methods involve administering a therapeutically effective amount of a dihydroorate dehydrogenase (DHODH) inhibitor to the subject.

In embodiments, administration of the DHODH inhibitor slows progression of ALS, reduces intensity of symptoms associated with ALS, delays onset of symptoms associated with ALS, reduces weight loss associated with ALS, reverses weight loss associated with ALS, delays mortality, or combinations thereof. Symptoms of ALS are well known. Such symptoms include, but are not limited to, symptoms affecting fine motor function, gross motor function, bulbar function, respiratory function, and combinations thereof (e.g. muscle twitching, muscle weakness, muscle control, walking, speech, eating, swallowing, writing, climbing stairs, cutting food, turning in bed, salivation, dressing, maintaining hygiene, breathing, dyspnea, orthopnea, respiratory insufficiency, and combinations thereof).

In some embodiments, administration of the DHODH inhibitor prevents or delays the onset of respiratory failure. In further embodiments, the method delays mortality associated with respiratory failure.

In aspects, the DHODH inhibitor is a small molecule chemical compound, antibody, nucleic acid molecule, polypeptide, or fragment thereof. In embodiments, the DHODH inhibitor inhibits biosynthesis of pyrimidine nucleotides. In embodiments, the DHODH inhibitor binds (e.g., specifically binds) to DHODH.

The DHODH inhibitor can be any DHODH inhibitor known in the art, including any DHODH described herein. In embodiments, the DHODH inhibitor is a substrate-like inhibitor; an isoxazolecarboxanilide or 3-hydroxy-2-cyanocrotanilide; a triazolopyrimidine based inhibitor; a trifluoromethy phenyl butenamide derivative; an ethoxy aromatic amide-based inhibitor; a cyclic aliphatic or aromatic carboxylic acid amide; an aromatic quinoline carboxamide derivative; a 2-phenylquinoline-4-carboxylic acid derivative; an aryl carboxylic acid amide derivative; a cyclopentene dicarboxylic acid amide derivative; a terphenyl carboxylic acid amide derivative; a cyclopropane carbonyl derivative; a biaryl carboxyamide derivative; a biphenyl-4-ylcarbamoyl thiophene/cyclopentene carboxylic acid derivative; an amino-benzoic acid derivative, an N-arylaminomethylene malonate derivative; a 4-hydroxycoumarin, fenamic acid or N-(alkylcarbonyl) anthranilic acid derivative; an alkyl-5-benzimidazole thiophene-2-carboxamide derivative; an amino nicotinic acid or isonicotinic acid derivative; or a salt thereof.

The present invention refers to a compound (Z)-2-cyano-3-hydroxy-but-2-enoic acid-(4′-trifluoromethylphenyl)-amide (teriflunomide) represented by the following structural formula,

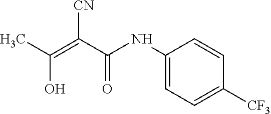 [SMILES: C/C(O)=C(\C#N)C(=O)Nc1ccc(C(F)(F)F)cc1]

wherein methods of using such compositions to treat subjects suffering from neurodegenerative diseases, such as ALS. Teriflunomide, an immunomodulatory agent with anti-inflammatory properties, inhibits dihydroorotate dehydrogenase, a mitochondrial enzyme involved in de novo pyrimidine synthesis. It is a white to almost white powder that is sparingly soluble in acetone, slightly soluble in polyethylene glycol and ethanol, very slightly soluble in isopropanol and practically insoluble in water.

The present invention also refers to a compound represented by the following structural formula,

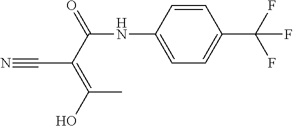 [SMILES: C/C(O)=C(/C#N)C(=O)Nc1ccc(C(F)(F)F)cc1]

wherein methods of using such compositions to treat subjects suffering from neurodegenerative diseases, such as ALS.

In embodiments, the DHODH inhibitor is (Z)-2-cyano-3-hydroxy-but-2-enoic acid-(4′-trifluoromethylphenyl)-amide or a salt thereof.

In aspects, the invention provides methods for delaying mortality in a human subject having or is at risk of having amyotrophic lateral sclerosis (ALS). In embodiments, the method involves administering a therapeutically effective amount of (Z)-2-cyano-3-hydroxy-but-2-enoic acid-(4′-trifluoromethylphenyl)-amide (teriflunomide) or a salt thereof to the subject. In some related embodiments, teriflunomide prevents or delays the onset of respiratory failure. In some related embodiments, teriflunomide delays mortality associated with respiratory failure.

In the above aspects and embodiments, the subject can be at risk of having ALS or may have been diagnosed with ALS. In some embodiments, the subject may not exhibiting symptoms of ALS.

In the above aspects and embodiments, the ALS can be familial ALS or sporadic ALS.

In the above aspects and embodiments, the subject can be a mammal (e.g., a human). In embodiments, the subject is an adult. In some embodiments, the subject is a female. In other embodiments, the subject is a male.

In the above aspects and embodiments, the DHODH inhibitor can be administered to the subject by any route (e.g., orally, topically, by inhalation, by injection, or the like). Such methods and routes are described in detail herein. In embodiments, the DHODH inhibitor is administered orally.

It is within the purview of the skilled artisan to determine an effective amount for use in the present invention. In some embodiments, the methods involve administering about 7 mg to about 14 mg of the DHODH inhibitor to the subject. In related embodiments, the methods involve administering 7 mg of the DHODH inhibitor to the subject. In other embodiments, the methods involve administering 14 mg of the DHODH inhibitor to the subject. In some embodiments, the methods involve administering about 0.001 mg/kg to about 100 mg/kg of the DHODH inhibitor to the subject. In further embodiments, the DHODH inhibitor is administered once daily.

In aspects, the above methods and embodiments can involve administering at least one additional agent to treat a symptom associated with ALS.

In aspects, the invention provides a dihydroorate dehydrogenase (DHODH) inhibitor for use in at least one of the methods described herein.

In aspects, the invention provides a composition containing a dihydroorate dehydrogenase (DHODH) inhibitor for use in at least one of the methods described herein. In related embodiments, the composition also contains a pharmaceutically acceptable carrier, diluent, or excipient. In yet another embodiment, the composition also contains at least one additional agent to treat a symptom associated with ALS.

In aspects, the invention provides a kit containing a dihydroorate dehydrogenase (DHODH) inhibitor for use in at least one of the methods described herein. In related embodiments, the composition also contains at least one additional agent to treat a symptom associated with ALS.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated herein and constitute a part of this specification, illustrate several embodiments of the invention and, together with the description, serve to explain the principles of the invention.

### DEFINITIONS

To facilitate an understanding of the present invention, a number of terms and phrases are defined below.

As used herein, the singular forms “a”, “an”, and “the” include plural forms unless the context clearly dictates otherwise.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease or a symptom thereof.

By “alteration” is meant a change (increase or decrease) in the expression levels or activity of a gene or polypeptide as detected by standard art known methods such as those described herein. As used herein, an alteration includes a 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more in expression levels.

As used herein, the term “amino” means a free radical having a nitrogen atom and 1 to 2 hydrogen atoms. As such, the term amino generally refers to primary and secondary amines. In that regard, as used herein and in the appended claims, a tertiary amine is represented by the general formula RR′N—, wherein R and R′ are carbon radicals that may or may not be identical. Nevertheless, the term “amino” generally may be used herein to describe a primary, secondary, or tertiary amine, and those of skill in the art will readily be able to ascertain the identification of which in view of the context in which this term is used in the present disclosure.

By “analog” is meant a molecule that is not identical, but has analogous functional or structural features. For example, a polypeptide analog retains at least some of the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term an “aromatic ring” or “aryl” means a monocyclic or polycyclic-aromatic ring or ring radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or optionally is substituted with one or more substituents, e.g., substituents as described herein for alkyl groups (including without limitation alkyl (preferably, lower alkyl or alkyl substituted with one or more halo), hydroxy, alkoxy (preferably, lower alkoxy), alkylthio, cyano, halo, amino, boronic acid (—B(OH)2, and nitro). In certain embodiments, the aryl group is a monocyclic ring, wherein the ring comprises 6 carbon atoms.

With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

“Colloidal silicon dioxide” is submicroscopic fumed silica, also known as pyrogenic silica. It is a non-crystalline, fine grain, low density and high surface area silica. Primary particle size is from 5 nm to 50 nm. The particles are non-porous and have a surface from 50 m2/g to 600 m2/g. It can be obtained for example under the trade name Aeorsil 200 Pharma from Evonik Industries [Evonik Degussa GmbH, Inorganic Materials, Weissfrauenstraβe 9, 60287 Frankfurt, Germany] or under the trade name CAB-O-SIL M-5P/5DP Cabot Corporation headquartered at Boston, Mass., U.S.A.

By “compound” is meant any small molecule chemical compound, antibody, nucleic acid molecule, or polypeptide, or fragments thereof.

“Degradant” refers to any drug-based materials generated after the preparation of the unit dosage form. Analysis of impurities and degradant is done using reverse phase HPLC techniques on extracted samples as is known in the art.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

The term “diastereomers” refers to stereoisomers with two or more centers of dissymmetry and whose molecules are not mirror images of one another.

The term “dihydroorotate dehydrogenase inhibitor” and “DHODH inhibitor” are used interchangeably and refer to an agent that reduces the intracellular pyrimidine pool in a cell. For example, the agent can inhibit biosynthesis of pyrimidine nucleotides by reducing dihydroorotate dehydrogenase activity (e.g., reducing oxidation of dihydroorotate to orotate).

By “disease” is meant any condition or disorder that damages or interferes with the normal function of a cell, tissue, or organ. Examples of diseases include neurodegenerative disorders, including ALS.

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “enantiomers” refers to two stereoisomers of a compound which are non-superimposable mirror images of one another. An equimolar mixture of two enantiomers is called a “racemic mixture” or a “racemate.”

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon. Heteroaryl groups may be optionally substituted with one or more substituents, e.g., substituents as described herein for aryl groups. Examples of heteroaryl groups include, but are not limited to, pyridyl, furanyl, benzodioxolyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, and indolyl.

The term “heteroatom” as used herein means an atom of any element other than carbon or hydrogen. Examples of heteroatoms include nitrogen, oxygen, sulfur and phosphorus. The term “isomers” or “stereoisomers” refers to compounds which have identical chemical constitution, but differ with regard to the arrangement of the atoms or groups in space.

The term “heterocyclic” as used herein, refers to organic compounds that contain at least at least one atom other than carbon (e.g., S, O, N) within a ring structure. The ring structure in these organic compounds can be either aromatic or, in certain embodiments, non-aromatic. Some examples of heterocyclic moeities include, are not limited to, pyridine, pyrimidine, pyrrolidine, furan, tetrahydrofuran, tetrahydrothiophene, and dioxane.

The term “hydroxyl” means —OH.

The term “isomers” or “stereoisomers” refers to compounds which have identical chemical constitution, but differ with regard to the arrangement of the atoms or groups in space.

The term “isotopic derivatives” includes derivatives of compounds in which one or more atoms in the compounds are replaced with corresponding isotopes of the atoms. For example, an isotopic derivative of a compound containing a carbon atom (C¹²) would be one in which the carbon atom of the compound is replaced with the C¹³ isotope.

As used herein, the term “neuroprotectant” refers to any agent that may prevent, ameliorate or slow the progression of neuronal degeneration and/or neuronal cell death.

“Pharmaceutically acceptable basic addition salt” is any non-toxic organic or inorganic basic addition salt (e.g., of the compound teriflunomide). Illustrative inorganic bases which form suitable salts include potassium hydroxide, sodium hydroxide, L-lysine or calcium hydroxide.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “polymorph” as used herein, refers to solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. Both configurations, cis/trans and/or Z/E are encompassed by the compounds of the present invention.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” or “increases” is meant a negative or positive alteration, respectively, of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% relative to a reference.

By “reference” is meant a standard or control condition.

By “subject” is meant a mammal, including, but not limited to, a human or non-human mammal, such as a bovine, equine, canine, ovine, or feline.

The term “sulfhydryl” or “thiol” means —SH.

As used herein, the term “tautomers” refers to isomers of organic molecules that readily interconvert by tautomerization, in which a hydrogen atom or proton migrates in the reaction, accompanied in some occasions by a switch of a single bond and an adjacent double bond.

As used herein, the terms “treat,” treating,” “treatment,” and the like refer to reducing or ameliorating a disorder and/or symptoms associated therewith. As described, by ameliorate is meant to decrease, suppress, attenuate, diminish, arrest, or stabilize the development or progression of a disease. It will be appreciated that, although not precluded, treating a disorder or condition does not require that the disorder, condition or symptoms associated therewith be completely eliminated.

Typically a the recommended dose of teriflunomide may be 7 mg or 14 mg taken orally once daily.

The phrase “combination therapy” embraces the administration of an agent described herein for the treatment of neurodegenerative diseases and a second therapeutic agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention comprises an agent described herein for the treatment of neurodegenerative diseases and at least one additional therapeutic agent (e.g., an agent for treating a symptom of the disease, including but not limited to, an antiglutamergic agent, a neuroprotective agent, an anti-inflammatory agent, an anti-apoptotic agent, a mitochondrial cofactor, an antioxidant, a copper chelating drug, a cyclo-oxygenase inhibitor, and the like) at the same or different times or they can be formulated as a single, co-formulated pharmaceutical composition comprising the two compounds. As another example, a combination of the present invention (e.g., an agent described herein for the treatment of neurodegenerative diseases and at least one additional therapeutic agent) is formulated as separate pharmaceutical compositions that can be administered at the same or different time. Sequential or substantially simultaneous administration of each therapeutic agent can be effected by any appropriate route including, but not limited to, oral routes, intravenous routes, intramuscular routes, and direct absorption through mucous membrane tissues (e.g., nasal, mouth, vaginal, and rectal). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present invention, exemplary methods, devices, and materials are now described. All technical and patent publications cited herein are incorporated herein by reference in their entirety. Nothing herein is to be construed as an admission that the invention is not entitled to antedate such disclosure by virtue of prior invention.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Additional features and advantages of compounds disclosed herein will be apparent from the following detailed description of certain embodiments.

### BRIEF DESCRIPTION OF THE DRAWINGS

FIG. 1 is a graph showing that teriflunomide significantly slows disease progression in symptomatic ALS mice following treatment with teriflunomide. 10 mg/kg of teriflunomide or vehicle (control) was administered by oral gavage to 82 day old SOD1-G93A mice (N=14 males/group). Control mice had a median survival of 127 days, and teriflunomide treatment extended median survival to 134 days (p<0.005).

FIGS. 2A and 2B are graphs showing that teriflunomide improves survival and functional outcomes in symptomatic ALS mice. 20 mg/kg of teriflunomide or vehicle (control) was administered by oral gavage to 82 day old male and female SOD1-G93A mice (N=14 mice/sex/group). As shown in FIG. 2A, teriflunomide treatment increased median survival from 130 days to 146.5 days (p<0.04). A grip strength performance test was used to assess grip strength. As shown in FIG. 2B, control mice had a 46.14-67.38% loss in grip strength as disease progressed. In contrast, teriflunomide treatment slowed muscle strength loss in both male and female SOD1-G93A mice to −20.14% and −24.06% in male and female mice, respectively.

FIGS. 3A and 3B are graphs showing that lymphocyte depletion does not alter disease course in ALS mice. SOD1-G93A mice were treated with vehicle or W19, an anti-CD52 mouse antibody. As shown in FIG. 3A, treatment with W19 depleted peripheral B cells, CD4⁺ cells, CD8⁺ cells, and NK cells. However, lymphocyte depletion did not significantly affect survival outcome of the SOD1-G93A mice. As shown in FIG. 3B, control mice and W19 treated mice had a median survival of 125 and 126 days, respectively (p=0.7575).

### DETAILED DESCRIPTION OF THE INVENTION

As described below, the present invention is based on the discovery that inhibitors of dihydroorotate dehydrogenase (DHODH) are effective in treating neurodegenerative diseases such as ALS. The invention features methods for using DHODH inhibitors to treat a subject having or at risk of having a neurodegenerative disease (e.g., ALS). The invention also features compositions for use in treating a subject having or at risk of having a neurodegenerative disease (e.g., ALS).

Amyotrophic lateral sclerosis (ALS) is a serious neurological disease that causes muscle weakness, disability and eventually death. ALS is often called Lou Gehrig's disease, after the famous baseball player who was diagnosed with it in 1939. In the U.S., ALS and motor neuron disease (MND) are sometimes used interchangeably. Worldwide, ALS occurs in 1 to 3 people per 100,000. In the vast majority of cases, which is known as sporadic form, —90 to 95 percent—doctors don't yet know why ALS occurs. About 5 to 10 percent of ALS cases are inherited. ALS often begins with muscle twitching and weakness in an arm or leg, or with slurring of speech. Eventually, ALS affects the ability to control the muscles needed to move, speak, eat and breathe. Early signs and symptoms of ALS include: difficulty lifting the front part of your foot and toes (footdrop), weakness in the leg, feet or ankles, hand weakness or clumsiness, slurring of speech or trouble swallowing, muscle cramps and twitching in your arms, shoulders and tongue. The disease frequently begins in the hands, feet or limbs, and then spreads to other parts of the body. As the disease advances, muscles become progressively weaker until they're paralyzed. It eventually affects chewing, swallowing, speaking and breathing. Without being limited as to theory, in ALS, the nerve cells that control the movement of the muscles gradually die, so the muscles progressively weaken and begin to waste away. Up to 1 in 10 cases of ALS is inherited. But the remainder appear to occur randomly.

ALS eventually paralyzes the muscles needed to breathe. The most common cause of death for people with ALS is respiratory failure, usually within three to five years after symptoms begin. When the muscles that control swallowing are affected, subjects with ALS can develop malnutrition and dehydration. They are also at higher risk of aspirating food, liquids or secretions into the lungs, which can cause pneumonia. Some subjects with ALS experience problems with memory and making decisions, and some are eventually diagnosed with a form of dementia called frontotemporal dementia.

The instant invention also relates to the use of DHODH inhibitors in a subject with ALS. Dihydroorotate dehydrogenase (DHODH) is an enzyme that catalyzes the oxidation of dihydroorotate to orotate, which is the fourth step of de novo pyrimidine biosynthesis. To date, DHODH inhibitors have been used for the treatment of cancer, parasitic infections, viral infections, and autoimmune disorders (e.g., multiple sclerosis). See Bratt, D. G. (1999) Expert Opin. Ther. Pat. 9:41-54; Christopherson, R. I. et al. (2002) Acc. Chem. Resh. 35:961-971; Löffler, M. et al. (2005) Trends Mol. Med. 11:430-437; Vyas, V. K. et al. (2011) Mini-Rev. Med. Chem. 11:1039-1055; and Munier-Lehmann, H. et al. (2013) J. Med. Chem. 56:3148-3167. The instant invention relates to the discovery that DHODH inhibitors are effective in subjects with neurodegenerative diseases such as ALS.

ALS subjects may be those with inherited ALS (familial ALS) or may be those with non-inherited ALS (sporadic ALS). Treatment of subjects with ALS using DHODH inhibitors may be be initiated prior to the onset of ALS symptoms (for example in patients with inherited forms of ALS) or may be initiated after the onset of ALS symptoms. ALS symptoms that may be ameliorated or prevented in subjects with ALS are muscle twitching, muscle weakness, muscle control, slurring of speech, respiratory failure, and lifespan of the subject with ALS. Early signs and symptoms of ALS that may be treated with DHODH inhibitors as exemplified herein, including teriflunomide (Z)-2-cyano-3-hydroxy-but-2-enoic acid-(4′-trifluoromethylphenyl)-amide (teriflunomide) as exemplified by the structure below. Early symptoms include: difficulty lifting the front part of your foot and toes (footdrop), weakness in the leg, feet or ankles, hand weakness or clumsiness, slurring of speech or trouble swallowing, muscle cramps and twitching in your arms, shoulders and tongue.

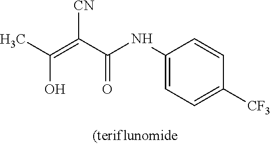 [SMILES: C/C(O)=C(\C#N)C(=O)Nc1ccc(C(F)(F)F)cc1]

Use of the compound represented by the following structural formula in the methods disclosed herein is also contemplated:

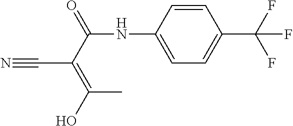 [SMILES: C/C(O)=C(/C#N)C(=O)Nc1ccc(C(F)(F)F)cc1]

wherein methods of using such compositions to treat subjects suffering from neurodegenerative diseases, such as ALS.

Although specific embodiments of the present disclosure will now be described, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### Dihydroorotate Dehydrogenase Inhibitors

Dihydroorotate dehydrogenase (DHODH) is an enzyme that catalyzes the oxidation of dihydroorotate to orotate, which is the fourth step of de novo pyrimidine biosynthesis. To date, DHODH inhibitors have been used for the treatment of cancer, parasitic infections, viral infections, and autoimmune disorders (e.g., multiple sclerosis). See Bratt, D. G. (1999) Expert Opin. Ther. Pat. 9:41-54; Christopherson, R. I. et al. (2002) Acc. Chem. Resh. 35:961-971; Löffler, M. et al. (2005) Trends Mol. Med. 11:430-437; Vyas, V. K. et al. (2011) Mini-Rev. Med. Chem. 11:1039-1055; and Munier-Lehmann, H. et al. (2013) J. Med. Chem. 56:3148-3167.

For example, a quinoline derivative, Brequinar, exhibits anticancer activity towards L1210 murine leukemia. See Andreson L. W. et al. (1989) Cancer Commun. 1:381-387; and Chen, S. F. et al. (1986) Cancer Res. 46:5014-5019. It has also been reported that Brequinar potentiates 5-fluorouracil antitumor activity in a murine colon 38 tumor model by tissue-specific modulation of uridine nucleotide pools. See Pizzorno, G. et al. (1992) Cancer Res. 52:1660-1665.

Use of DHODH inhibitors as antibiotics against parasites has also been proposed. For example, DHODH inhibitors may be useful against Helicobacter pylori (see, e.g., Marcinkeviciene et al. (2000) Biochem. Pharmacol. 60:339; and Haque, T. S. et al. (2002) J. Med. Chem. 45:4669-4678) and Plasmodium falciparum (see, e.g., Heikkilä, T. et al. (2007) J. Med. Chem. 50:186-191; Heikkilä, T. et al. (2006) Bioorg. Med. Chem. Lett. 16:88-92; Cassera, M. B. et al. (2011) Curr. Top. Med. Chem. 11:2103-2115; and Phillips, M. A. et al. (2010) Infect. Disord.: Drug Targets 10:226-239).

DHODH inhibitors can be useful as antifungal agents (see, e.g., Gustafson, G. et al. (1996) Curr. Genet. 30:159-165) or to treat viral mediated diseases (see, e.g., U.S. Pat. No. 6,841,561)

Furthermore, DHODH inhibition may be useful in treating transplant rejection, rheumatoid arthritis, psoriasis, as well as autoimmune diseases, including multiple sclerosis. See Kovarik, J. M. et al. (2003) Expert Opin. Emerg. Drugs 8:47-62; Allison, A. C. (1993) Transplantation Proc. 25(3) Suppl. 2:8-18); Makowka, L. (1993) Immunolog. Rev. 136:51-70; Davis J. P. et al. (1996) Biochemistry 35:1270-1273; Boyd, B. et al. (2005) Drugs Future 30:1102-1106; O'Connor, P. W. et al. (2006) Neurology 66:894-9000; Palmer, A. M. (2010) Curr. Opin. Invest. Drugs 11:1313-1323; and Claussen, M. C. et al. (2012) 142:49-56.

Surprisingly, it has now been discovered that DHODH inhibitors are effective against neurodegenerative diseases such as ALS.

As DHODH inhibitors and methods for making and using DHODH inhibitors are well known in the art, it is within the purview of the skilled artisan to use any DHODH inhibitor in the methods described herein. The following description provides several embodiments of the invention, and it is to be understood that these examples are not restrictive of the invention.

In aspects, the DHODH inhibitor is a substrate-like inhibitor (e.g., a pyrimidine related to the substrate or the product of the reaction or a quinone related to the ubiquinone co-factor) (see Batt, D. G. (1999) Exp. Opin. Ther. Patents 9:41-54; and Defrees, S. A. et al. (1988) Biochem. Pharmacol. 37:3807-3816); a cinchoninic acid derivative (see Dexter, D. L. et al. (1985) Cancer Res. 45:5563-5568; EP 133244; U.S. Pat. No. 4,680,299; U.S. Pat. No. 5,032,597; EP 339485; U.S. Pat. No. 4,861,783; WO 9119498; and WO 9742953); an isoxazolecarboxanilide or 3-hydroxy-2-cyanocrotanilide (see Munier-Lehmann, H. et al. (2013) J. Med. Chem. 56:3148-3167); a triazolopyrimidine based inhibitor (see Phillips, M. A. et al. (2008) J. Med. Chem. 51:3649-3653; Gujjar, R. et al. (2009) J. Med. Chem. 52: 1864-1872; Phillips, M. A. et al. (2010) Infectious Disorders—Drug Targets 10:226-239; and Deng, X. et al. (2009) J. Biol. Chem. 284:26999-27009); a trifluoromethy phenyl butenamide derivative (see Davies, M. et al. (2009) J. Med. Chem. 52: 2683-2693); an ethoxy aromatic amide-based inhibitor (see Heikkila, T. et al. (2007) J. Med. Chem. 50:186-191); a cyclic aliphatic or aromatic carboxylic acid amide derivative (see Baumgartner, R. et al. (2006) J. Med. Chem. 49:1239-1247; and Leban, J. et al. (2004) Bioorg. Med. Chem. Lett. 14:55-58); an aromatic quinoline carboxamide derivative (see Papageorgiou, C. et al. (2001) J. Med. Chem. 44:1986-1992); a 2-phenylquinoline-4-carboxylic acid derivative (see Boa, A. N. et al. (2005) 13:1945-1967); an aryl carboxylic acid amide derivative (see Vyas, V. K. et al. (2012) Ind. J. Chem. 51B:1749-1760); a cyclopentene dicarboxylic acid amide derivative (see Leban, J. et al. (2005) Bioorg. Med. Chem. Lett. 15:4854-4857); a terphenyl carboxylic acid amide derivative (see Sutton, A. E. et al. (2001) 42:547-557; and Hurt, D. E. et al. (2006) Bioorg. Med. Chem. Lett. 16:1610-1615); a cyclopropane carbonyl derivative (see Kuo, P. Y. et al. (2006) Bioorg. Med. Chem. Lett. 16:6024-6027); a biaryl carboxyamide derivative (see Heikkila, T. et al. (2006) Bioorg. Med. Chem. Lett. 16:88-92); a biphenyl-4-ylcarbamoyl thiophene/cyclopentene carboxylic acid derivative (see Leban, J. et al. (2006) Bioorg. Med. Chem. Lett. 16:267-270); an amino-benzoic acid derivative (see McLean, L. R. et al. (2010) Bioorg. Med. Chem. Lett. 20:1981-1984); an N-arylaminomethylene malonate derivative (see Cowen, D. et al. (2010) Bioorg. Med. Chem. Lett. 20:1284-1287); a 4-hydroxycoumarin, fenamic acid or N-(alkylcarbonyl) anthranilic acid derivative (see Fritzson, I. et al. (2010) Chem. Med. Chem. 5:608-617); an alkyl-5-benzimidazole thiophene-2-carboxamide derivative (see Booker, M. L. et al. (2010) J. Biol. Chem. 285:33054-33064; and Patel, V. et al. (2008) J. Biol. Chem. 283:35078-35085); or an amino nicotinic acid or isonicotinic acid derivative (See International Patent Publication No. WO2008077639). See Vyas, V. K. et al. (2011) Mini-Reviews Med. Chem. 11:1039-1055.

In embodiments, the DHODH inhibitor is a substrate-like inhibitor. In some embodiments, the DHODH inhibitor is 5-aza-dihydroorotate; cis-5-methyldihydroorotate; orotate; a spirobarbiturate, a hydantoin, lapachol, dichloroallyl lawsone, BW58c, or atovaquone. In some embodiments, the DHODH inhibitor is

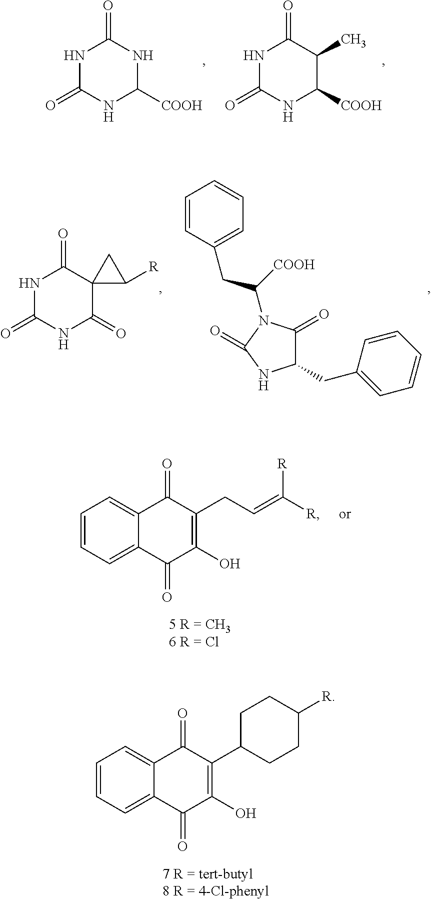

In embodiments, the DHODH inhibitor is a cinchoninic acid derivative. In some embodiments, the DHODH inhibitor is Brequinar, a Brequinar analog, or a Brequinar derivative. See Slobada, A. E. et al. (1991) J. Rheumatol. 18:855-860; Ito, T. et al. (1997) Organ Biol. 4:43-48; Nakajima, H. et al. (1997) Organ Biol. 4:49-57; Pitts, W. J. et al. (1998) Bioorg. Med. Chem. Lett. 8:307-312; Jacobson, I. C. et al. (1998) 216^{th} ACS Meeting ORGN132; Batt, D. G. et al. (1998) 8:1745-1750; WO 9429478; U.S. Pat. No. 4,639,454; JP803163A; EP 305952; EP 379145; U.S. Pat. No. 4,918,077; U.S. Pat. No. 5,002,954; WO 9200739; WO 9506640; U.S. Pat. No. 5,371,225; EP 721942; JP 10231289; JP 6306079 A2; and U.S. Pat. No. 5,523,408. In related embodiments, the DHODH inhibitor is

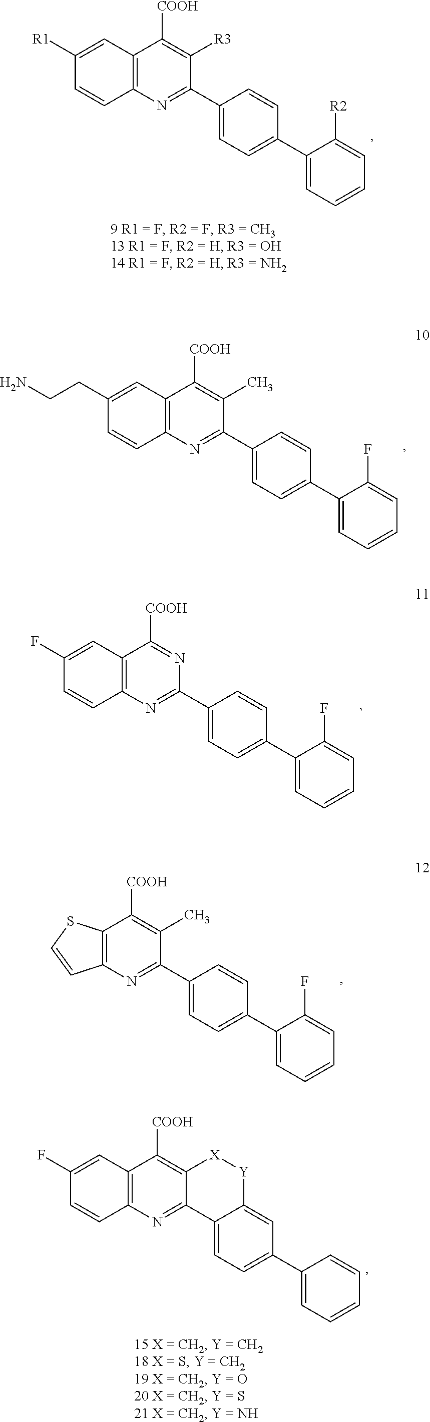

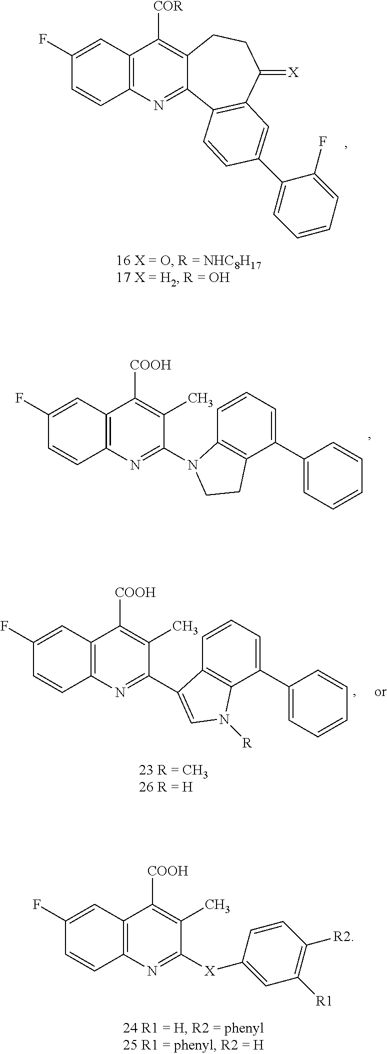

In some embodiments, the DHODH inhibitor is

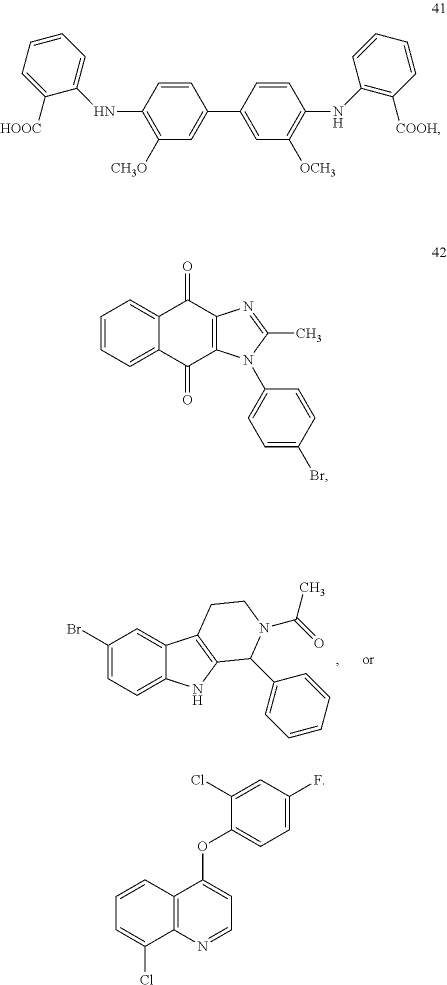

In embodiments, the DHODH inhibitor is isoxazolecarboxanilide, 3-hydroxy-2-cyanocrotanilide, or an analog/derivative thereof. See Munier-Lehmann, H. et al. (2013) J. Med. Chem. 56:3148-3167; Kuo, E. A. et al. (1996) J. Med. Chem. 39:4608-4621; Albert, R. et al. (1998) Bioorg. Med. Chem. Lett. 8:2203-2208; Bertolini, G. et al. (1997) J. Med. Chem. 40:2011-2016; Papageorgiou, C. et al. (1997) 25:233-238; Ren, S. et al. (1998) 15:286-295; DE 2524929; WO 9117748; EP 538783; EP 257882; EP 259972; EP 484223; EP 646578; EP 551230; EP 533573; EP 632017; EP 652214; EP 606175; EP 767167; EP 573318; EP 829470; or WO 9424095. In some embodiments, the DHODH inhibitor is

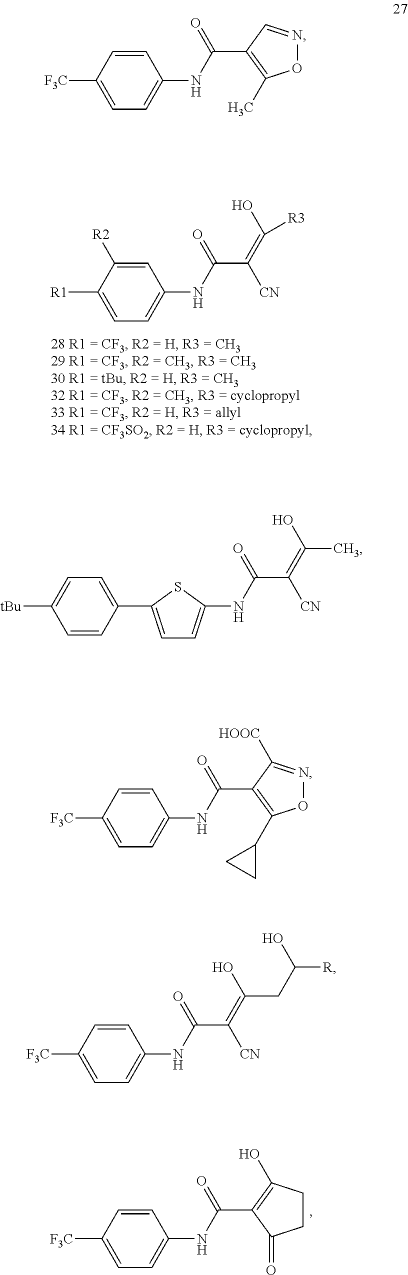

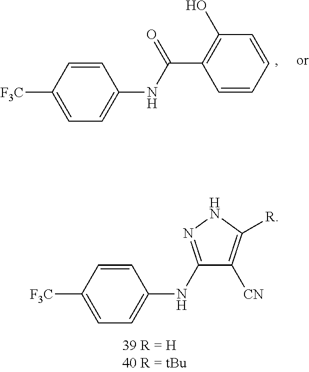

In embodiments, the DHODH inhibitor is a triazolopyrimidine based inhibitor. See Phillips, M. A. et al. (2008) J. Med. Chem. 51:3649-3653; Gujjar, R. et al. (2009) J. Med. Chem. 52: 1864-1872; Phillips, M. A. et al. (2010) Infectious Disorders—Drug Targets 10:226-239; and Deng, X. et al. (2009) J. Biol. Chem. 284:26999-27009. In some embodiments, the DHODH inhibitor is

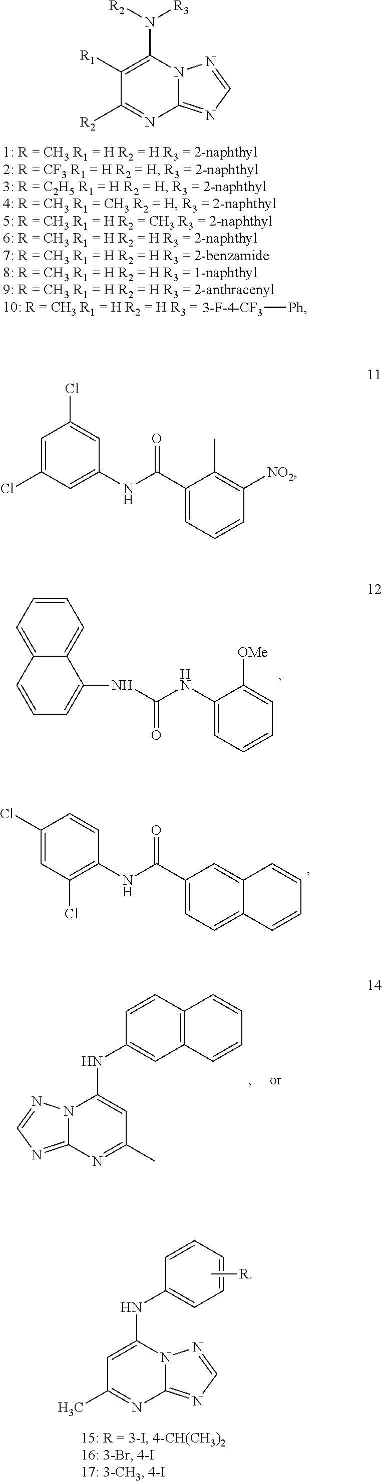

In embodiments, the DHODH inhibitor is a trifluoromethy phenyl butenamide derivative. See Davies, M. et al. (2009) J. Med. Chem. 52: 2683-2693. In some embodiments, the DHODH inhibitor is

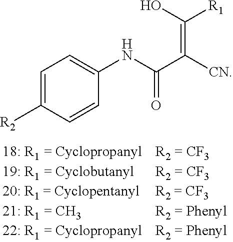

In embodiments, the DHODH inhibitor is an ethoxy aromatic amide-based inhibitor. See Heikkila, T. et al. (2007) J. Med. Chem. 50:186-191. In some embodiments, the DHODH inhibitor is

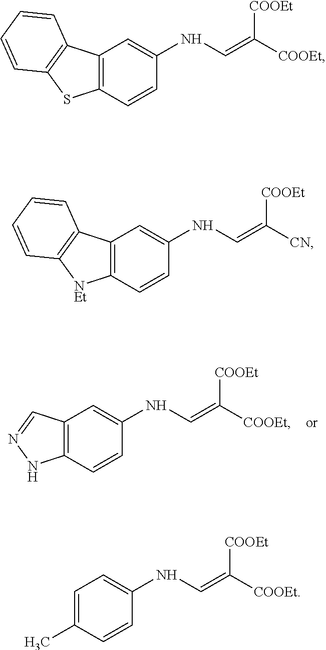

In embodiments, the DHODH inhibitor is a cyclic aliphatic or aromatic carboxylic acid amide derivative. See Baumgartner, R. et al. (2006) J. Med. Chem. 49:1239-1247; and Leban, J. et al. (2004) Bioorg. Med. Chem. Lett. 14:55-58. In some embodiments, the DHODH inhibitor is

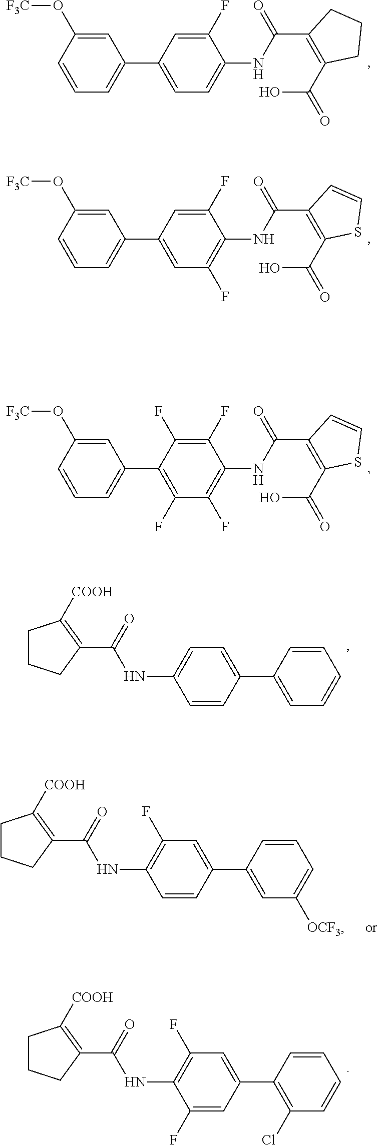

In embodiments, the DHODH inhibitor is an aromatic quinoline carboxamide derivative. See Papageorgiou, C. et al. (2001) J. Med. Chem. 44:1986-1992. In some embodiments, the DHODH inhibitor is

| 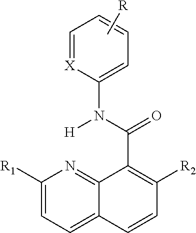 | | | | |
|---|---|---|---|---|
| Comp. | R | R₁ | R₂ | X |
| 33 | 4-CF₃ | H | H | C |
| 34 | 4-CF3 | H | H | N |
| 35 | H | H | H | C |
| 36 | 2-CF₃ | H | H | C |

In embodiments, the DHODH inhibitor is a 2-phenylquinoline-4-carboxylic acid derivative. See Boa, A. N. et al. (2005) 13:1945-1967. In some embodiments, the DHODH inhibitor is

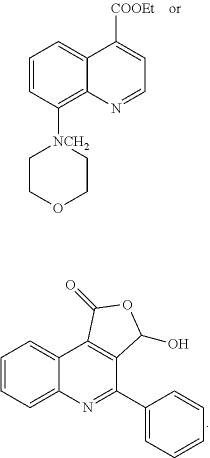

In embodiments, the DHODH inhibitor is an aryl carboxylic acid amide derivative. See Vyas, V. K. et al. (2012) Ind. J. Chem. 51B:1749-1760.

In embodiments, the DHODH inhibitor is a cyclopentene dicarboxylic acid amide derivative. See Leban, J. et al. (2005) Bioorg. Med. Chem. Lett. 15:4854-4857. In some embodiments, the DHODH inhibitor is

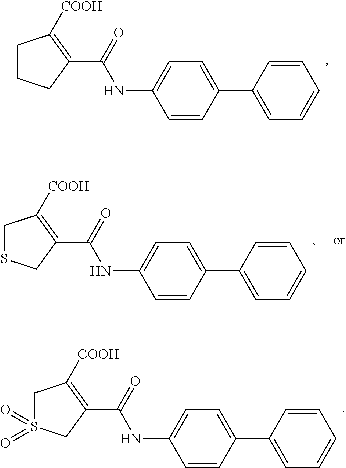 [SMILES: O=C(O)C1=C(C(=O)Nc2ccc(-c3ccccc3)cc2)CCC1 ; O=C(O)C1=C(C(=O)Nc2ccc(-c3ccccc3)cc2)CSC1 ; O=C(O)C1=C(C(=O)Nc2ccc(-c3ccccc3)cc2)CS(=O)(=O)C1]

In embodiments, the DHODH inhibitor is a terphenyl carboxylic acid amide derivative. See Sutton, A. E. et al. (2001) 42:547-557; and Hurt, D. E. et al. (2006) Bioorg. Med. Chem. Lett. 16:1610-1615. In some embodiments, the DHODH inhibitor is

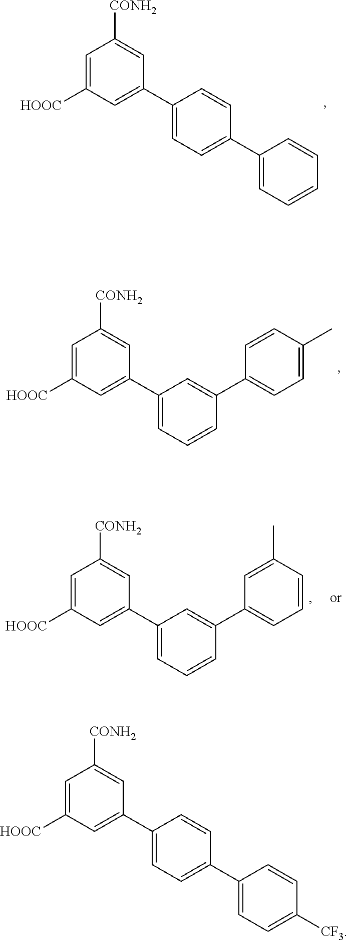 [SMILES: NC(=O)c1cc(C(=O)O)cc(-c2ccc(-c3ccccc3)cc2)c1 ; Cc1ccc(-c2cccc(-c3cc(C(N)=O)cc(C(=O)O)c3)c2)cc1 ; Cc1cccc(-c2cccc(-c3cc(C(N)=O)cc(C(=O)O)c3)c2)c1 ; NC(=O)c1cc(C(=O)O)cc(-c2ccc(-c3ccc(C(F)(F)F)cc3)cc2)c1]

In embodiments, the DHODH inhibitor is a cyclopropane carbonyl derivative. See Kuo, P. Y. et al. (2006) Bioorg. Med. Chem. Lett. 16:6024-6027. In some embodiments, the DHODH inhibitor is

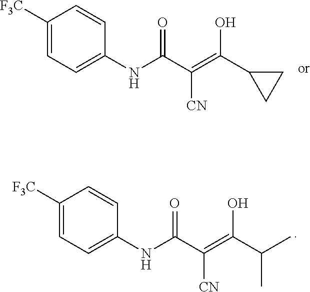 [SMILES: N#C/C(C(=O)Nc1ccc(C(F)(F)F)cc1)=C(/O)C1CC1 ; CC(C)/C(O)=C(\C#N)C(=O)Nc1ccc(C(F)(F)F)cc1]

In embodiments, the DHODH inhibitor is a biaryl carboxyamide derivative. See Heikkila, T. et al. (2006) Bioorg. Med. Chem. Lett. 16:88-92. In some embodiments, the DHODH inhibitor is

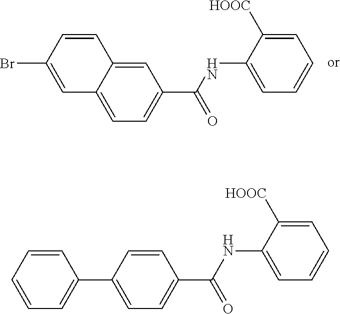 [SMILES: O=C(Nc1ccccc1C(=O)O)c1ccc2cc(Br)ccc2c1 ; O=C(Nc1ccccc1C(=O)O)c1ccc(-c2ccccc2)cc1]

In embodiments, the DHODH inhibitor is a biphenyl-4-ylcarbamoyl thiophene/cyclopentene carboxylic acid derivative. See Leban, J. et al. (2006) Bioorg. Med. Chem. Lett. 16:267-270. In some embodiments, the DHODH inhibitor is

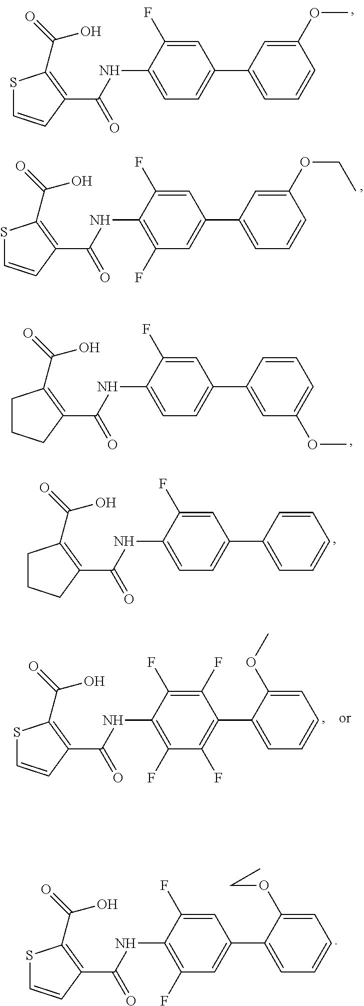

In embodiments, the DHODH inhibitor is an amino-benzoic acid derivative. See McLean, L. R. et al. (2010) Bioorg. Med. Chem. Lett. 20:1981-1984. In some embodiments, the DHODH inhibitor is

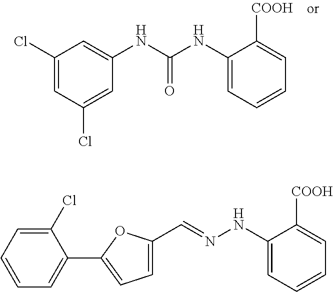 [SMILES: O=C(Nc1cc(Cl)cc(Cl)c1)Nc1ccccc1C(=O)O ; O=C(O)c1ccccc1N/N=C/c1ccc(-c2ccccc2Cl)o1]

In embodiments, the DHODH inhibitor is an N-arylaminomethylene malonate derivative. See Cowen, D. et al. (2010) Bioorg. Med. Chem. Lett. 20:1284-1287. In some embodiments, the DHODH inhibitor is

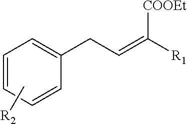

| Compound | R1 | R2 | PfDHODH(50 μM) | hDHODH(50 μM) |
|---|---|---|---|---|
| 58 | CO₂Et | m-CO₂Et | 0 ± 4 | 5 ± 2 |
| 59 | CO₂Et | p-CO₂Et | 15 ± 4 | 4 ± 2 |
| 60 | CN | p-CO₂Et | 2 ± 4 | 2 ± 2 |

In embodiments, the DHODH inhibitor is a 4-hydroxycoumarin, fenamic acid or N-(alkylcarbonyl) anthranilic acid derivative. See Fritzson, I. et al. (2010) Chem. Med. Chem. 5:608-617. In some embodiments, the DHODH inhibitor is

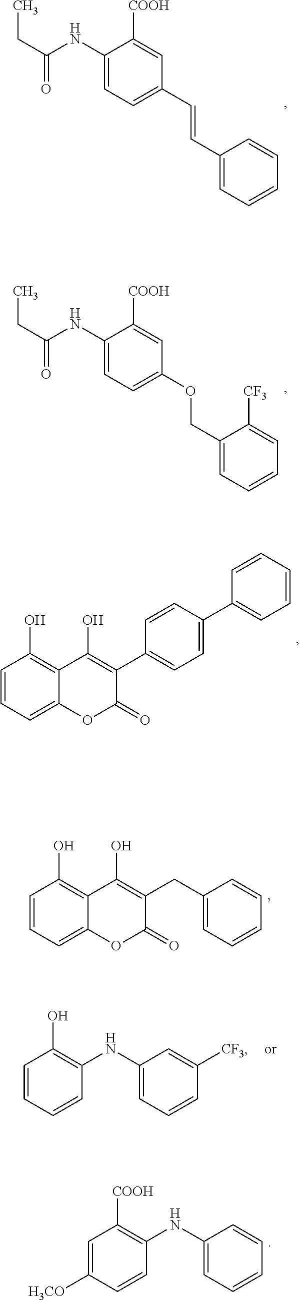

In embodiments, the DHODH inhibitor is an alkyl-5-benzimidazole thiophene-2-carboxamide derivative. See Booker, M. L. et al. (2010) J. Biol. Chem. 285:33054-33064; and Patel, V. et al. (2008) J. Biol. Chem. 283:35078-35085. In some embodiments, the DHODH inhibitor is

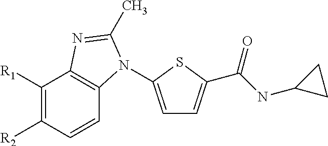

| Comp. | R₁ | R₂ |
|---|---|---|
| 73 | H | OCF₃ |
| 74 | OCHF₃ | H |
| 75 | CN | H |

In embodiments, the DHODH inhibitor is an amino nicotinic acid or isonicotinic acid derivative. See International Patent Publication No. WO 2008077639. In some embodiments, the DHODH inhibitor is

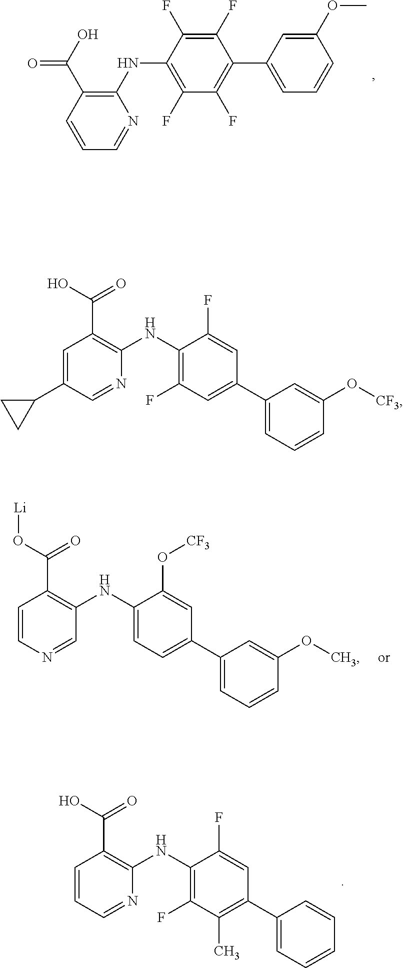

In embodiments, the DHODH inhibitor is (Z)-2-cyano-3-hydroxy-but-2-enoic acid-(4′-trifluoromethylphenyl)-amide (teriflunomide) represented by the following structural formula,

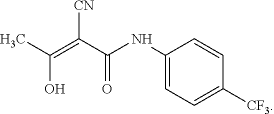 [SMILES: C/C(O)=C(\C#N)C(=O)Nc1ccc(C(F)(F)F)cc1]

Teriflunomide is the generic name for the compound (Z)-2-cyano-3-hydroxy-but-2-enoic acid-(4′-trifluoromethylphenyl)-amide. Teriflunomide can be used in the form in which it is chemically prepared, or it can be subjected to a process which changes the physical nature of the particles. For example, the material can be milled by any process known in the art. Non-exclusive examples of such processes include mechanical milling and jet milling. The particles produced either directly from the process of chemically preparing teriflunomide or after a milling operation preferably provide average particle diameters in the range of 1 μm to 100 μm. It is advantageous to use said teriflunomide particles from 1 μm to 100 μm in the preparation of the solid pharmaceutical composition, especially at about 1% to 10% weight:weight of teriflunomide. The synthesis of teriflunomide has been disclosed, and is accomplished by methods that are well known to those skilled in the art. For example, U.S. Pat. No. 5,990,141, issued on Nov. 23, 1999 discloses methods of synthesis. The dosage range at which teriflunomide exhibits its ability to act therapeutically can vary depending upon its severity, the patient, other underlying disease states that the patient is suffering from, and other medications that may be concurrently administered to the patient. Generally, teriflunomide will exhibit their therapeutic activities at dosages of between about 0.001 mg/kg of patient body weight/day to about 100 mg/kg of patient body weight/day.

In embodiments, the DHODH inhibitor is a compound represented by the following structural formula,

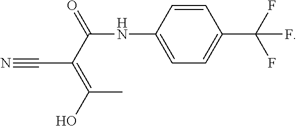 [SMILES: C/C(O)=C(/C#N)C(=O)Nc1ccc(C(F)(F)F)cc1]

In embodiments, the DHODH inhibitor is cis-4-carboxy-6-(mercaptomethyl)-3,4,5,6-tetrahydropyrimidin-2(1H)-one; 2-acetyl-6-bromo-1-phenyl-2,3,4,8-tetrahydro-1H-pyrido[3,4-b]indole; 2-cyano-3-cyclopropyl-3-hydroxy-N-[4-(trifluoromethylsulfonyl)phenyl]-2-propenamide; 2-cyano-3-cyclopropyl-3-hydroxy-N-[4-(trifluoromethylsulfinyl)phenyl]-2-propenamide; (+)-cis-6-oxo-4-sulfanyl-1,4-azaphosphorinane-2-carboxylic acid P-oxide; (+)-trans-6-Oxo-4-sulfanyl-1,4-azaphosphorinane-2-carboxylic acid P-oxide; 3,5-DHBA (3,5-dihydroxybenzoic acid); N3-benzyl-1-(4-chlorophenyl)-N5-phenyl-1H-pyrazole-3,5-dicarboxamide; 1-(4-chlorophenyl)-N5-phenyl-N3-(pyridin-3-ylmethyl)-1H-pyrazole-3,5-dicarboxamide; 2-[N-(4-biphenylyl)carbamoyl]-1-cyclopentene-1-carboxylic acid; 3′-methoxy-2′,5′,6′-trimethyl-4′-[6-(3-methyl-2-butenylamino)pyridin-3-yl]-4-(3-methyl-2-butenyloxy)biphenyl-3-ol; 2′,5′-dimethoxy-3′,6′-dimethyl-4-(3-methyl-2-butenyloxy)-4′-[6-(3-methyl-2-butenyloxy)pyridin-3-yl]biphenyl-3-ol; 3′-methoxy-2′,5′,6′-trimethyl-4-(3-methyl-2-butenyloxy)-4′-[6-(3-methyl-2-butenyloxy)pyridin-3-yl]biphenyl-3-ol; 2-[N-[2,3,5,6-tetrafluoro-3′-(trifluoromethoxy)biphenyl-4-yl]carbamoyl]-1-cyclopentene-1-carboxylic acid; 2-[N-(2,3,5,6-tetrafluoro-2′-methoxybiphenyl-4-yl)carbamoyl]-1-cyclopentene-1-carboxylic acid; 2-[N-(3,5-difluoro-2′-methoxybiphenyl-4-yl)carbamoyl]-1-cyclopentene-1-carboxylic acid; 2[N-[3,5-difluoro-3′-(trifluoromethoxy)biphenyl-4-yl]carbamoyl]-1-cyclopentene-1-carboxylic acid; 3-hydroxy-2-[N-[2,3,5,6-tetrafluoro-3′-(trifluoromethoxy)biphenyl-4-yl]carbamoyl]-1-cyclopentene-1-carboxylic acid; 5-hydroxy-2-[N-[2,3,5,6-tetrafluoro-3′-(trifluoromethoxy)biphenyl-4-yl]carbamoyl]-1-cyclopentene-1-carboxylic acid; 2-[N-(3′-ethoxy-3,5-difluorobiphenyl-4-yl)carbamoyl]-3-hydroxy-1-cyclopentene-1-carboxylic acid; 2-[N-(3′-Ethoxy-3,5-difluorobiphenyl-4-yl)carbamoyl]-5-hydroxy-1-cyclopentene-1-carboxylic acid; 3-hydroxy-2-[N-(2′,3,5-trifluorobiphenyl-4-yOcarbamoyl]-1-cyclopentene-1-carboxylic acid; 5-hydroxy-2-[N-(2′,3,5-trifluorobiphenyl-4-yl)carbamoyl]-1-cyclopentene-1-carboxylic acid; 2-[N-(2-Chloro-2′-methoxybiphenyl-4-yl)carbamoyl]-3-hydroxy-1-cyclopentene-1-carboxylic acid; 2-[N-(2-chloro-2′-methoxybiphenyl-4-yl)carbamoyl]-5-hydroxy-1-cyclopentene-1-carboxylic acid; 2-[N-(2′-chloro-3,5-difluorobiphenyl-4-yl)carbamoyl]-3-hydroxy-1-cyclopentene-1-carboxylic acid; 2-[N-(2′-chloro-3,5-difluorobiphenyl-4-yl)carbamoyl]-5-hydroxy-1-cyclopentene-1-carboxylic acid; 2-[N-(3-fluoro-3′-methoxybiphenyl-4-yl)carbamoyl]-3-hydroxy-1-cyclopentene-1-carboxylic acid; 2-[N-(3-fluoro-3′-methoxybiphenyl-4-yl)carbamoyl]-5-hydroxy-1-cyclopentene-1-carboxylic acid; 3-[N-(4-biphenylyl)carbamoyl]thiophene-2-carboxylic acid; 3-[N-(2′-ethoxy-3,5-difluorobiphenyl-4-yl)carbamoyl]thiophene-2-carboxylic acid; 3-[N-(2,3,5,6-tetrafluoro-2′-methoxybiphenyl-4-yl)carbamoyl]thiophene-2-carboxylic acid; 3-[N-(2-chloro-2′-methoxybiphenyl-4-yl)carbamoyl]thiophene-2-carboxylic acid; 3-[N-[3,5-difluoro-3′-(trifluoromethoxy)biphenyl-4-yl]carbamoyl]thiophene-2-carboxylic acid; 4-[N-(2-chloro-2′-methoxybiphenyl-4-yl)carbamoyl]thiophene-3-carboxylic acid; 4-[N-(2′-ethoxy-3,5-difluorobiphenyl-4-yl)carbamoyl]thiophene-3-carboxylic acid; 4-[N-(3-fluoro-3′-methoxybiphenyl-4-yl)carbamoyl]thiophene-3-carboxylic acid; 4-[N-(4-biphenylyl)carbamoyl]thiophene-3-carboxylic acid; 2-[N-(4-biphenylyl)carbamoyl]furan-3-carboxylic acid; 2-propionamido-5-[2-(trifluoromethyl)phenylsulfanyl]benzoic acid; 2-propionamido-5-[2-(trifluoromethyl)benzyloxy]benzoic acid; 2-propionamido-5-[2-(trifluoromethyl)phenoxymethyl]benzoic acid; 2-propionamido-5-[2-(trifluoromethyl)phenoxy]benzoic acid; 4-[N-[2,3,5,6-tetrafluoro-3′-(trifluoromethoxy)biphenyl-4-yl]carbamoyl]-2,5-dihydrothiophene-3-carboxylic acid; N-[4-[(E)-2-phenylvinyl]phenyl]-D-prolinamide hydrochloride; N-[4-[2-[4-(trifluoromethoxy)phenyl]ethynyl]phenyl]-D-prolinamide hydrochloride; N-[4-[2-(4-benzylphenyl)ethynyl]phenyl]-D-prolinamide hydrochloride; N-(4-Octylphenyl)-D-prolinamide hydrochloride; N-[4-(cyclohexylmethylsulfanyl)phenyl]-D-prolinamide hydrochloride; 5-[4′-(isopropylamino)-2,5-dimethylbiphenyl-4-ylmethylene]thiazolidine-2,4-dione; 5-[4′-(isobutylamino)-2,5-dimethylbiphenyl-4-ylmethylene]thiazolidine-2,4-dione; 5-[2′-fluoro-4′-(isopropylamino)-2,5-dimethylbiphenyl-4-ylmethylene]oxazolidine-2,4-dione; 5-[2′-fluoro-4′-(2-furylmethylamino)-2,5-dimethylbiphenyl-4-ylmethylene]oxazolidine-2,4-dione; 6-[2-fluoro-4-(isopropylamino)phenyl]-N-isopropylnaphthalene-2-carboxamide; 6-[2-fluoro-4-(isopropylamino)phenyl]naphthalene-2-carboxylic acid; 7-[2-fluoro-4-(isopropylamino)phenyl]-N-isopropyl-8-methyl-2-oxo-1,2-dihydroquinoline-3-carboxamide; N-cyclopentyl-6-[2-fluoro-4-(isopropylamino)phenyl]naphthalene-2-carboxamide; N-cyclopropyl-6-[2-fluoro-4-(isopropylamino)phenyl]naphthalene-2-carboxamide; 2(R)-[6-[2-fluoro-4-(isopropylamino)phenyl]naphthalene-2-carboxamido]propionic acid methyl ester; 6-[2-fluoro-4-(isopropylamino)phenyl]-N-isopropyl-1,5-dimethoxynaphthalene-2-carboxamide; 2-[2-fluoro-4-(isopropylamino)phenyl]-N-isopropylquinoline-6-carboxamide; 2-(2,3,5,6-tetrafluoro-3′-methoxybiphenyl-4-ylamino)pyridine-3-carboxylic acid; 2-(3,5-difluoro-2-methylbiphenyl-4-ylamino)pyridine-3-carboxylic acid; 5-cyclopropyl-2-[3,5-difluoro-3′-(trifluoromethoxy)biphenyl-4-ylamino]pyridine-3-carboxylic acid; 2-(2′-chloro-3,5-difluorobiphenyl-4-ylamino)-5-cyclopropylpyridine-3-carboxylic acid; 2-(2′-chloro-3,5-difluoro-2-methylbiphenyl-4-ylamino)pyridine-3-carboxylic acid; 5-[2-(2,4-dichlorophenyl)ethenyl]-2-(propionamido)benzoic acid; 5-[2-(2-chloro-4-fluorophenyl)ethenyl]-2-(propionamido)benzoic acid; 5-[2-[4-chloro-2-(trifluoromethyl)phenyl]ethenyl]-2-(propionamido)benzoic acid; 5-cyclopropyl-2-[6-phenyl-5-(trifluoromethyl)pyridin-3-ylamino]benzoic acid; 5-cyclopropyl-2-[5-methyl-6-[3-(trifluoromethoxy)phenyl]pyridin-3-ylamino]benzoic acid; 2-[6-(2-fluorophenyl)-5-methylpyridin-3-ylamino]-5-methylbenzoic acid; 2-[6-(2-chlorophenyl)pyridin-3-ylamino]-5-cyclopropylbenzoic acid; 2-[2-(2-chlorophenyl)pyrimidin-5-ylamino]-5-cyclopropylbenzoic acid; N-[2′-chloro-3-(trifluoromethyl)biphenyl-4-yl]-2-cyano-3-hydroxy-2-butenamide; 2-cyano-N-(2′,3-dichlorobiphenyl-4-yl)-3-hydroxy-2-butenamide; 2-cyano-3-hydroxy-N-(2′,3,6′-trichlorobiphenyl-4-yl)-2-butenamide; 2-[3,5-difluoro-2-methyl-3′-(trifluoromethoxy)biphenyl-4-ylamino]pyridine-3-carboxylic acid; 2-(3,5-difluoro-2′-methylbiphenyl-4-ylamino)pyridine-3-carboxylic acid; 5-[3-fluoro-3′-(trifluoromethoxy)biphenyl-4-ylamino]-2-methylpyridine-4-carboxylic acid; 5-cyclopropyl-2-(5-fluoro-3′-methoxy-2-methylbiphenyl-4-ylamino)pyridine-3-carboxylic acid; 2-(cyclopropylcarboxamido)-5-[N-methyl-N-(pyridin-3-ylmethyl)amino]benzoic acid; 2-[2-[5-(2-chlorophenyl)furan-2-ylmethylene]hydrazino]benzoic acid; 2-[3-(3,5-Dichlorophenyl)ureido]benzoic acid; 2-(biphenyl-4-ylamino)-5-methoxybenzoic acid; 5-[2(E)-phenylvinyl]-2-propionamidobenzoic acid; 5-cyclopropyl-2-[2-(2,6-difluorophenyl)pyrimidin-5-ylamino]benzoic acid sodium salt; 5-cyclopropyl-2-[2-(2,6-difluorophenyl)pyrimidin-5-ylamino]benzoic acid tromethamine salt; 2-(3,5-difluoro-3′-methoxybiphenyl-4-ylamino)pyridine-3-carboxylic acid N-methyl-D-glucamine salt; 2-[3′-ethoxy-3-(trifluoromethoxy)biphenyl-4-ylamino]pyridine-3-carboxylic acid N-methyl-D-glucamine salt; 2-(3,5-difluoro-2-methylbiphenyl-4-ylamino)pyridine-3-carboxylic acid tromethamine salt; 2-(3,5-difluoro-3′-methoxybiphenyl-4-ylamino)pyridine-3-carboxylic acid tromethamine salt; 2-(3,5-difluoro-3′-methoxybiphenyl-4-ylamino)pyridine-3-carboxylic acid L-arginine salt; 2-(2,3,3′,5,5′,6-hexafluorobiphenyl-4-yl)-6-methyl-1H-benzimidazole-4-carboxylic acid; 6-methyl-2-(2,2′,3,5,6-pentafluorobiphenyl-4-yl)-1H-benzimidazole-4-carboxylic acid; 1,5-dimethyl-2-(2,3,5,6-tetrafluorobiphenyl-4-yl)-1H-benzimidazole-7-carboxylic acid; 2-(2′,3-difluorobiphenyl-4-yl)-5-methyl-1H-benzimidazole-7-carboxylic acid; 2-(2′-fluorobiphenyl-4-yl)-5-methyl-1H-benzimidazole-7-carboxylic acid; 6-methyl-2-(2,3,5,6-tetrafluorobiphenyl-4-yl)-1H-benzimidazole-4-carboxylic acid; 5-(anthracen-9-ylmethyl)hexahydropyrimidine-2,4,6-trione; 5-[4-(benzyloxy)-2-hydroxybenzylidene]hexahydropyrimidine-2,4,6-trione; 5-(pyren-1-ylmethyl)hexahydropyrimidin-2,4,6-trione; 5-(phenanthren-9-ylmethyl)hexahydropyrimidin-2,4,6-trione; 5-(5-nitrofuran-2-ylmethyl)hexahydropyrimidin-2,4,6-trione; 5-(5-nitrofuran-2-ylmethylene)hexahydropyrimidin-2,4,6-trione; 5-[3-(5-nitrofuran-2-yl)-2-propenyl]hexahydropyrimidin-2,4,6-trione; 5-[3-(5-nitrofuran-2-yl)-2-propenylidene]hexahydropyrimidin-2,4,6-trione; 6-fluoro-2-(4-phenoxyphenyl)quinoline-4-carboxylic acid; 2-[N-(3′-butoxy-3-chloro-5-fluorobiphenyl-4-yl)carbamoyl]benzoic acid; 2-[N-(3′-ethoxy-3,5-difluorobiphenyl-4-yl)carbamoyl]benzoic acid; 2-[N-(3-chloro-5-fluoro-3′-propoxybiphenyl-4-yl)carbamoyl]benzoic acid; 2-[N-(3-chloro-5-fluoro-3′-isobutoxybiphenyl-4-yl)carbamoyl]benzoic acid; 2-[N-(3′-butoxy-3,5-difluorobiphenyl-4-yl)carbamoyl]benzoic acid; 2-[N-[3-chloro-3′-(cyclopentyloxy)-5-fluorobiphenyl-4-yl]carbamoyl]benzoic acid; 4-(2-cyano-3-hydroxy-2-pentenoylamino)-2′,4′-difluorobiphenyl-2-carboxylic acid methyl ester; 2-[N-(3-fluoro-3′-methoxybiphenyl-4-yl)carbamoyl]-1-cyclopentenecarboxylic acid N-methyl-D-glucamine salt; 2-[N-(3-fluoro-3′-methoxybiphenyl-4-yl)carbamoyl]-1-cyclopentenecarboxylic acid diethylamine salt; 4-hydroxy-N-[2,3,5,6-tetrafluoro-3′-(trifluoromethoxy)biphenyl-4-yl]-1,2,5-oxadiazole-3-carboxamide; 5-[3-(4-Chlorophenyl)-3-oxoprop-1-en-1-yl]-2-hydroxybenzoic acid; 4-[(naphthalen-2-ylmethylidene)amino]phenol; 1-[2-[(3-chloro-4-methylphenyl)amino]-4-methyl-1,3-thiazol-5-yl]ethanone; ethyl 4-phenyl-2-(5,6,7,8-tetrahydronaphthalen-2-ylamino)-1,3-thiazole-5-carboxylate; ethyl 2-[(3,4-dimethylphenyl)amino]-4-phenyl-1,3-thiazole-5-carboxylate; ethyl 2-[(3-chloro-4-methylphenyl)amino]-4-phenyl-1,3-thiazole-5-carboxylate; ethyl 2-[(3-fluoro-4-methylphenyl)amino]-4-phenyl-1,3-thiazole-5-carboxylate; ethyl 2-(2,3-dihydro-1H-inden-5-ylamino)-4-phenyl-1,3-thiazole-5-carboxylate; ethyl 2-(2-naphthylamino)-4-oxo-4,5-dihydrofuran-3-carboxylate; Ethyl 2-(2-naphthylamino)-4-oxo-4,5-dihydrothiophene-3-carboxylate; N-(1,3-benzothiazol-2-yl)-3-(trichloromethyl)benzamide; N-(1,3-benzothiazol-2-yl)-3-hydroxy-4-methoxybenzamide; N-(1,3-Benzothiazol-2-yl)-3-fluorobenzamide; N-(1,3-benzothiazol-2-yl)-2,4-dichlorobenzamide; N-(4,6-dimethyl-1,3-benzothiazol-2-yl)-3-ethoxybenzamide; 3-Methoxy-N-(6-methyl-1,3-benzothiazol-2-yl)benzamide; 2-[[[4-(4-methylphenyl)-1,3-thiazol-2-yl]hydrazono]methyl]phenol; 2-[[(4-phenyl-1,3-thiazol-2-yl)hydrazono]methyl]benzaldehyde; 2-[N-[4-(4-bromophenyl)-1,3-thiazol-2-yl]ethanehydrazonoyl]phenol; 3-[[4-(3-hydroxyphenyl)-1,3-thiazol-2-yl]hydrazono]indan-1-one; 2-[[[4-(4-chlorophenyl)-1,3-thiazol-2-yl]hydrazono]methyl]phenol; 4-(4-bromophenyl)-2-[2-(2-furylmethylene)hydrazino]-1,3-thiazole; 2-ethoxy-4-[[(5-methyl-4-phenyl-1,3-thiazol-2-yl)hydrazono]methyl]phenol; or 6-Fluoro-2-[2-methyl-4-phenoxy-5-(propan-2-yl)phenyl]quinoline-4-carboxylic acid.

In embodiments, the DHODH inhibitor is leflunomide (5-methyl-N-[4-(trifluoromethyl)phenyl]isoxazole-4-carboxamide); protocatechuic acid (3,4-dihydroxybenzoic acid); manitimus (2-cyano-3-hydroxy-N-[4-(trifluoromethyl)phenyl]hepta-2-en-6-ynamide); AB-22405 or ABR-224050 (Chelsea Therapeutics; Active Biotech); ASLAN-003 or LAS-186323 (Almirall; ASLAN Pharmaceuticals); vidofludimus (2-[N-(3-Fluoro-3′-methoxybiphenyl-4-yl)carbamoyl]-1-cyclopentene-1-carboxylic acid); 2-cyano-N-(4-cyanophenyl)-3-cyclopropyl-3-hydroxyacrylamide; 10-fluoro-3-(2-fluorophenyl)-6,7-dihydro-5H-benz[6,7]cyclohepta[1,2-b]quinoline-8-carboxylic acid; N-[2-Fluoro-2′,5′-dimethyl-4′-[6-(3-methyl-2-butenyloxy)pyridin-3-yl]biphenyl-4-yl]-N-(3-methyl-2-butenyl)amine; LAS-187247 (Almirall); 4-(2-cyano-3-hydroxy-2-pentenoylamino)-4′-fluorobiphenyl-2-carboxylic acid methyl ester; or RP-7047 (Rhizen Pharmaceuticals; Incozen).

### Methods of Treatment

The invention includes methods for treating a subject having or at risk of having amyotrophic lateral sclerosis (ALS).

In aspects, the invention provides methods for treating ALS in a subject by administering a therapeutically effective amount of a dihydroorate dehydrogenase (DHODH) inhibitor to the subject.

In aspects, the invention provides methods for delaying mortality in a by administering a therapeutically effective amount of a dihydroorate dehydrogenase (DHODH) inhibitor to the subject.

In embodiments, administration of the DHODH inhibitor slows progression of ALS, reduces intensity of symptoms associated with ALS, delays onset of symptoms associated with ALS, reduces weight loss associated with ALS, reverses weight loss associated with ALS, delays mortality, or combinations thereof.

In embodiments, administration of the DHODH inhibitor prevents or delays the onset of respiratory failure. In some embodiments, the method delays mortality associated with respiratory failure.

In aspects, the invention provides methods for delaying mortality in a human subject by administering a therapeutically effective amount of (Z)-2-cyano-3-hydroxy-but-2-enoic acid-(4′-trifluoromethylphenyl)-amide (teriflunomide) or a salt thereof to the subject. In some related embodiments, teriflunomide prevents or delays the onset of respiratory failure. In some related embodiments, teriflunomide delays mortality associated with respiratory failure.

### Pharmaceutical Compositions

The invention provides for compositions containing at least one agent described herein for the treatment of neurodegenerative diseases. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing neurodegenerative disease. A thorough discussion of pharmaceutically acceptable carriers, diluents, and other excipients is presented in Remington's Pharmaceutical Sciences (20th ed., Mack Publishing Co. 2000), which is hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof. Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions. Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder. In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container.

Methods for formulating the pharmaceutical compositions of the present invention are conventional and well known in the art (see Remington's). One of skill in the art can readily formulate a pharmaceutical composition having the desired characteristics (e.g., route of administration, biosafety, release profile, and the like).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th ed., Lippincott Williams & Wilkins), which is hereby incorporated by reference.

### Formulation

Teriflunomide Formulations.

Teriflunomide is also known as (Z)-2-cyano-3-hydroxy-but-2-enoic acid-(4′-trifluoromethylphenyl)-amide. Teriflunomide can be used in the form in which it is chemically prepared, or it can be subjected to a process which changes the physical nature of the particles. For example, the material can be milled by any process known in the art. Non-exclusive examples of such processes include mechanical milling and jet milling. The particles produced either directly from the process of chemically preparing teriflunomide or after a milling operation preferably provide average particle diameters in the range of 1 μm to 100 μm. It is advantageous to use said teriflunomide particles from 1 μm to 100 μm in the preparation of the solid pharmaceutical composition, especially at about 1% to 10% weight:weight of teriflunomide.

Teriflunomide can be a solid pharmaceutical composition comprising:
a) about 1% to 30% weight:weight Teriflunomide, or a pharmaceutically acceptable basic addition salt thereof,
b) about 5% to 20% weight:weight disintegrant,
c) about 0% to 40% weight:weight binder,
d) about 0.1% to 2% weight:weight lubricant and
e) the remaining percentage comprising diluents,

provided that said solid pharmaceutical composition does not contain colloidal silicon dioxide.

A formulation can be a solid pharmaceutical composition comprising about 1% to 30% weight:weight (w:w) teriflunomide, or a pharmaceutically acceptable basic addition salt thereof, about 5% to 20% weight:weight disintegrant, about 0% to 40% weight:weight binder, about 0.1% to 2% weight:weight lubricant and the remaining percentage comprising diluents provided that said solid pharmaceutical composition does not contain colloidal silicon dioxide. A second aspect is a solid pharmaceutical composition comprising about 1% to 20% weight:weight teriflunomide, or a pharmaceutically acceptable basic addition salt thereof, about 5% to 20% weight:weight disintegrant, about 0% to 30% weight:weight binder, about 0.1% to 2% weight:weight lubricant, about 1% to 20% weight:weight acidic reacting compound and the remaining percentage comprising diluents. A third aspect is a solid pharmaceutical composition comprising about 1% to 20% weight:weight teriflunomide, or a pharmaceutically acceptable basic addition salt thereof, about 5% to 20% weight:weight disintegrant, about 0% to 30% weight:weight binder, about 0.1% to 2% weight:weight lubricant, about 1% to 20% weight:weight acidic reacting compound, about 0.1% to 0.5% weight:weight colloidal silicon dioxide and the remaining percentage comprising diluents.

Teriflunomide can be a solid pharmaceutical composition comprising a therapeutically effective amount of teriflunomide or a pharmaceutically acceptable basic addition salt thereof, wherein the solid pharmaceutical composition does not contain colloidal silicon dioxide. It may also be in a solid pharmaceutical composition comprising from about 2% to 15% weight:weight teriflunomide and the other components disintegrant, binder, lubricant and diluents show the same amount as defined under b) to e) above. It may also be a solid pharmaceutical composition comprising from about 7% to 15% weight:weight disintegrant and the other components teriflunomide, binder, lubricant and diluents show the same amount as defined under a) and c) to e) above. A formulation can be a solid pharmaceutical composition comprising from about 15% to 35% weight:weight binder and the other components teriflunomide, disintegrant, lubricant and diluents show the same amount as defined under a), b), d) and e) above.

The teriflunomide solid pharmaceutical composition can be one comprising from about 0.1% to 1.0% weight:weight lubricant and the other components teriflunomide, disintegrant, binder and diluents show the same amount as defined under a) to c) and e) above.

Examples of disintegrants are carboxymethylcellulose, low substituted hydroxypropyl cellulose, microcrystalline cellulose, powdered cellulose, croscarmellose sodium, methylcellulose, polacrilin potassium, sodium alginate, sodium starch glycolate or a mixture of one or more of said disintegrants. Examples of binders are acacia, carboxymethylcellulose, hydroxyethylcellulose, hydroxypropylcellulose, dextrin, gelatin, guar gum, hydroxypropyl methylcellulose, maltodextrin, methylcellulose, sodium alginate, pregelatinized starch, starches such as potato starch, corn starch or cereal starch and zein or a mixture of one or more of said binders. Examples of lubricants are calcium stearate, glyceryl palmitostearate, sodium benzoate, sodium lauryl sulfate, sodium stearyl fumarate, stearic acid, talc, zinc stearate and magnesium stearate or a mixture of one or more of said lubricants. Examples of diluents are cellulose, cellulose acetate, dextrates, dextrin, dextrose, fructose, 1-O-α-D-Glucopyranosyl-D-mannitol, glyceryl palmitostearate, hydrogenated vegetable oil, kaolin, lactitol, lactose, lactose mono-hydrate, maltitol, mannitol, maltodextrin, maltose, pregelatinized starch, sodium chloride, sorbitol, starches, sucrose, talc and xylitol or a mixture of one or more of said diluents.

A solid pharmaceutical composition can be one comprising from 2% to 15% weight:weight teriflunomide, 7% to 15% weight:weight disintegrant selected from one or more of microcrystalline cellulose or sodium starch glycolate, 15% to 35% weight:weight binder selected from one or more of hydroxypropylcellulose or corn starch, 0.1% to 1.0% weight:weight lubricant selected from magnesium stearate and the remaining percentage comprising diluents selected from lactose mono-hydrate.

Furthermore, the solid pharmaceutical composition can comprise:

A) about 1% to 20% weight:weight teriflunomide, or a pharmaceutically acceptable basic addition salt thereof,

B) about 5% to 20% weight:weight disintegrant,

C) about 0% to 30% weight:weight binder,

D) about 0.1% to 2% weight:weight lubricant,

E) about 1% to 20% weight:weight acidic reacting compound and

F) the remaining percentage comprising diluents.

In addition, the formulation can be a solid pharmaceutical composition comprising from about 2% to 15% weight:weight teriflunomide and the other components disintegrant, binder, lubricant, acidic reacting compound and diluents show the same amount as defined under B) to F) above. The formulation can be a solid pharmaceutical composition comprising from about 7% to 15% weight:weight disintegrant and the other components teriflunomide, binder, lubricant, acidic reacting compound and diluents show the same amount as defined under A) and C) to F) above. It can be a solid pharmaceutical composition comprising from about 15% to 30% weight:weight binder and the other components teriflunomide, disintegrant, lubricant, acidic reacting compound and diluents show the same amount as defined under A), B) and D) to F) above. It can be a solid pharmaceutical composition comprising from about 0.1% to 1.0% weight:weight lubricant and the other components teriflunomide, disintegrant, binder, acidic reacting compound and diluents show the same amount as defined under A) to C), E and F) above. It can be a solid pharmaceutical composition comprising from about 3% to 20% weight:weight acidic reacting compound and the other components teriflunomide, disintegrant, binder, lubricant and diluents show the same amount as defined under A) to D) and F) above.

Examples of acidic reacting compound are citric acid, acetic acid, glycolic acid, lactic acid, pyruvic acid, malonic acid, succinic acid, glutaric acid, fumaric acid, malic acid, tartaric acid, ascorbic acid, maleic acid, hydroxymaleic acid, benzoic acid, hydroxybenzoic acid, phenylacetic acid, cinnamic acid, salicyclic acid, 2-phenoxybenzoic acid, p-toluenesulfonic acid and sulfonic acids such as methanesulfonic acid and 2-hydroxyethanesulfonic acid or a mixture of one or more of said acidic reacting compound.

Teriflunomide is mixed with said disintegrant, binder, lubricant and diluents constituents to obtain the concentration of teriflunomide and said further components according to the present invention in the final mixture and finally is mixed with an acidic reacting compound. In addition, the formulation can be a solid pharmaceutical composition comprising components A) to F) as defined above shows a pH from 4.5 to 2.0, when water is adsorbed to the pharmaceutical composition or when water is added in small amounts to the pharmaceutical composition. In a further embodiment of the invention the solid pharmaceutical composition comprising components A) to F) as defined above shows a pH from about pH 3 to about pH 2. The pH determination is performed by suspending one tablet in about 1 ml of purified water. The pH of the supernatant is determined with a pH sensitive probe.

In a further embodiment, the formulation can be a solid pharmaceutical composition comprising a therapeutically effective amount of teriflunomide or a pharmaceutically acceptable basic addition salt thereof, wherein the pH of the solid pharmaceutical composition is less than about 4.5, particularly from about 4.5 to about 2.0, more particularly from about 3 to about 2.

The solid pharmaceutical composition may be:

A) about 1% to 20% weight:weight teriflunomide, or a pharmaceutically acceptable basic addition salt thereof,

B) about 5% to 20% weight:weight disintegrant,

C) about 0% to 30% weight:weight binder,

D) about 0.1% to 2% weight:weight lubricant,

E) about 1% to 20% weight:weight acidic reacting compound,

F) about 0.1% to 0.5% weight:weight colloidal silicon dioxide and

G) the remaining percentage comprising diluents.

It may be a solid pharmaceutical composition comprising from about 2% to 15% weight:weight teriflunomide and the other components disintegrant, binder, lubricant, acidic reacting compound, colloidal silicon dioxide and diluents show the same amount as defined under B) to G) above. It may be a solid pharmaceutical composition comprising from about 7% to 15% weight:weight disintegrant and the other components teriflunomide, binder, lubricant, acidic reacting compound, colloidal silicon dioxide and diluents show the same amount as defined under A) and C) to G) above. It may be a solid pharmaceutical composition comprising from about 15% to 30% weight:weight binder and the other components teriflunomide, disintegrant, lubricant, acidic reacting compound, colloidal silicon dioxide and diluents show the same amount as defined under A), B) and D) to G) above. It may be, a solid pharmaceutical composition comprising from about 0.1% to 1.0% weight:weight lubricant and the other components teriflunomide, disintegrant, binder, acidic reacting compound, colloidal silicon dioxide and diluents show the same amount as defined under A) to C), E and G) above. It may be a solid pharmaceutical composition comprising from about 3% to 20% weight:weight acidic reacting compound and the other components teriflunomide, disintegrant, binder, lubricant, colloidal silicon dioxide and diluents show the same amount as defined under A) to D) and F) and G) above. It may a solid pharmaceutical composition comprising from about 0.2% to 0.4% weight:weight colloidal silicon dioxide and the other components teriflunomide, disintegrant, binder, lubricant, acidic reacting compound and diluents show the same amount as defined under A) to E) and G) above. It may be a solid pharmaceutical composition comprising from about 0.3% weight:weight colloidal silicon dioxide and the other components teriflunomide, disintegrant, binder, lubricant, acidic reacting compound and diluents show the same amount as defined under A) to E) and G) above.

Teriflunomide is mixed with said disintegrant, binder, lubricant, colloidal silicon dioxide and diluents constituents to obtain the concentration of teriflunomide and said further components according to the present invention in the final mixture and finally is mixed with an acidic reacting compound. In a further embodiment of the invention the solid pharmaceutical composition comprising components A) to G) as defined above shows a pH from 4.5 to 2.0, when water is adsorbed to the pharmaceutical composition or when water is added in small amounts to the pharmaceutical composition. In a further embodiment of the invention the solid pharmaceutical composition comprising components A) to G) as defined above shows a pH from about pH 3 to about pH 2.

In providing teriflunomide formulations in forms suitable for unit dosage formation, the teriflunomide and the further components of the solid pharmaceutical composition according to the invention can be mixed as powders. This mixing can be carried out using any of the mixing techniques known in the art. The mixing is may be carried out using a high shear mixer, V-blender (or other twin-shell blender), bin blender or Turbula mixer-shaker. Blending is typically carried out first without the addition of a lubricant for sufficient time to assure complete mixing. At that point, the lubricant is typically added followed by a short (about 1-10 minute) further mixing period. Once the blend is made, unit dosage forms are prepared by procedures known in the art. In one aspect, unit dosage forms are made on rotary tablet presses or capsule filling machines. The dosage forms thus prepared can then optionally be coated with a film designed to provide ease of swallowing, a proprietary or identification appearance and/or protection of the dosage form.

Alternatively, additional processes for preparing a wet granulation of teriflunomide and the further components of the solid pharmaceutical composition comprise the following steps:

(a) blending of the teriflunomide with diluent and optionally some or all of the remaining excipients needed for the final composition. These other excipients can include binders, disintegrant, lubricant, acidic reacting compound and colloidal silica;

(b) adding a granulation solvent while the material from step (a) is under shear. Preferred granulation solvents include, water, ethanol, isopropanol and combinations thereof. Other ingredients can be added to the granulation solvent as known in the art. Examples of such additives are binders, acidic reacting compounds, wetting agents, stabilizers and buffers. The solvent can be applied by any technique known in the art. Preferred methods of applying the solvent while imparting shear include high shear granulation, low shear granulation, fluid bed granulation and extrusion granulation;

(c) optionally, the material from step (b) can be milled, ground or sieved. This wet material is then dried, preferably using air drying, fluid bed drying, oven drying or microwave drying. The drying is preferably carried out such that the drying temperature does not exceed about 60° C.;

(d) optionally this material is then milled or sieved;

(e) the material is then blended with additional excipients; and

(f) the composition is optionally formed into a unit dosage form, preferably a tablet or a capsule.

The dosage forms thus prepared can then optionally be coated with a film designed to provide ease of swallowing, a proprietary or identification appearance and/or protection of the dosage form.

In a further example, the solid pharmaceutical composition may comprise a therapeutically effective amount of teriflunomide or a pharmaceutically acceptable basic addition salt thereof, wherein the solid pharmaceutical composition contains no more than about 0.1%, or particularly no more than about 0.05% by weight of 2-cyano-N-(4-trifluoromethyl-phenyl)-acetamide after being stored at about 25° C. and about 65% relative humidity for about 12 months.

In a further example, the solid pharmaceutical composition comprising a therapeutically effective amount of teriflunomide or a pharmaceutically acceptable basic addition salt thereof, wherein the solid pharmaceutical composition contains no more than about 0.3%, or particularly no more than about 0.2%, or more particularly no more than about 0.05% by weight of 2-cyano-N-(4-trifluoromethyl-phenyl)-acetamide after being stored at about 25° C. and about 65% relative humidity for about 36 months.

In a further example, the solid pharmaceutical composition comprising a therapeutically effective amount of teriflunomide or a pharmaceutically acceptable basic addition salt thereof, wherein the solid pharmaceutical composition contains no more than about 0.3%, particularly no more than about 0.1%, or more particularly no more than about 0.05% by weight of 2-cyano-N-(4-trifluoromethyl-phenyl)-acetamide after being stored at about 30° C. and about 65% relative humidity for about 12 months.

In a further example, the solid pharmaceutical composition comprising a therapeutically effective amount of teriflunomide or a pharmaceutically acceptable basic addition salt thereof, wherein the solid pharmaceutical composition contains no more than about 1%, or particularly no more than about 0.5%, or more particularly no more than about 0.05% by weight of 2-cyano-N-(4-trifluoromethyl-phenyl)-acetamide after being stored at about 30° C. and about 65% relative humidity for about 36 months.

In a further example, the solid pharmaceutical composition comprising a therapeutically effective amount of teriflunomide or a pharmaceutically acceptable basic addition salt thereof, wherein the solid pharmaceutical composition contains no more than about 0.3%, particularly no more than about 0.1%, or more particularly no more than about 0.05% by weight of 2-cyano-N-(4-trifluoromethyl-phenyl)-acetamide after being stored at about 30° C. and about 75% relative humidity for about 12 months.

### Methods of Delivery

The pharmaceutical compositions of the invention can be administered to a subject by oral and non-oral means (e.g., topically, transdermally, or by injection). Such modes of administration and the methods for preparing an appropriate pharmaceutical composition for use therein are described in Gibaldi's Drug Delivery Systems in Pharmaceutical Care (1st ed., American Society of Health-System Pharmacists), which is hereby incorporated by reference.

In embodiments, the pharmaceutical compositions are administered orally in a solid form.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent. The tablets and other solid dosage forms, such as dragees, capsules, pills, and granules, can optionally be scored or prepared with coatings and shells, such as enteric coatings and other coatings well known in the art.

The pharmaceutical compositions can also be formulated so as to provide slow, extended, or controlled release of the active ingredient therein using, for example, hydroxypropylmethyl cellulose in varying proportions to provide the desired release profile, other polymer matrices, liposomes and/or microspheres. The pharmaceutical compositions can also optionally contain opacifying agents and may be of a composition that releases the active ingredient(s) only, or preferentially, in a certain portion of the gastrointestinal tract, optionally, in a delayed manner. Examples of embedding compositions include polymeric substances and waxes. The active ingredient can also be in micro-encapsulated form, if appropriate, with one or more pharmaceutically acceptable carriers, excipients, or diluents well known in the art (see, e.g., Remington's).

In embodiments, the pharmaceutical compositions are administered orally in a liquid form. Liquid dosage forms for oral administration of an active ingredient include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., cottonseed, groundnut, corn, germ, olive, castor and sesame oils), glycerol, tetrahydrofuryl alcohol, polyethylene glycols and fatty acid esters of sorbitan, and mixtures thereof. In addition to inert diluents, the liquid pharmaceutical compositions can include adjuvants such as wetting agents, emulsifying and suspending agents, sweetening, flavoring, coloring, perfuming and preservative agents, and the like.

Suspensions, in addition to the active ingredient(s) can contain suspending agents such as, but not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof.

In embodiments, the pharmaceutical compositions are administered by non-oral means such as by topical application, transdermal application, injection, and the like. In related embodiments, the pharmaceutical compositions are administered parenterally by injection, infusion, or implantation (e.g., intravenous, intramuscular, intra-arterial, subcutaneous, and the like).

Compositions for parenteral use can be presented in unit dosage forms, e.g., in ampoules or in vials containing several doses, and in which a suitable preservative can be added. Such compositions can be in form of a solution, a suspension, an emulsion, an infusion device, a delivery device for implantation, or it can be presented as a dry powder to be reconstituted with water or another suitable vehicle before use. One or more co-vehicles, such as ethanol, can also be employed. Apart from the active ingredient(s), the compositions can contain suitable parenterally acceptable carriers and/or excipients or the active ingredient(s) can be incorporated into microspheres, microcapsules, nanoparticles, liposomes, or the like for controlled release. Furthermore, the compositions can also contain suspending, solubilising, stabilising, pH-adjusting agents, and/or dispersing agents.

The pharmaceutical compositions can be in the form of sterile injections. The pharmaceutical compositions can be sterilized by, for example, filtration through a bacteria-retaining filter, or by incorporating sterilizing agents in the form of sterile solid compositions which can be dissolved in sterile water, or some other sterile injectable medium immediately before use. To prepare such a composition, the active ingredient is dissolved or suspended in a parenterally acceptable liquid vehicle. Exemplary vehicles and solvents include, but are not limited to, water, water adjusted to a suitable pH by addition of an appropriate amount of hydrochloric acid, sodium hydroxide or a suitable buffer, 1,3-butanediol, Ringer's solution and isotonic sodium chloride solution. The pharmaceutical composition can also contain one or more preservatives, for example, methyl, ethyl or n-propyl p-hydroxybenzoate. To improve solubility, a dissolution enhancing or solubilising agent can be added or the solvent can contain 10-60% w/w of propylene glycol or the like.

The pharmaceutical compositions can contain one or more pharmaceutically acceptable sterile isotonic aqueous or nonaqueous solutions, dispersions, suspensions or emulsions, or sterile powders, which can be reconstituted into sterile injectable solutions or dispersions just prior to use. Such pharmaceutical compositions can contain antioxidants; buffers; bacteriostats; solutes, which render the formulation isotonic with the blood of the intended recipient; suspending agents; thickening agents; preservatives; and the like.

Examples of suitable aqueous and nonaqueous carriers, which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include, but are not limited to, biodegradable/bioerodible polymers such as polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

In embodiments, the active ingredient(s) are administered by aerosol. This is accomplished by preparing an aqueous aerosol, liposomal preparation, or solid particles containing the compound. A nonaqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions. Dosage forms for topical or transdermal administration of an active ingredient(s) includes powders, sprays, ointments, pastes, creams, lotions, gels, solutions, patches, inhalants, and the like. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249; 4,906,169; 5,198,223; 4,816,540; 5,422,119; and 5,023,084, which are hereby incorporated by reference. The transdermal patch can also be any transdermal patch well known in the art, including transscrotal patches. Pharmaceutical compositions in such transdermal patches can contain one or more absorption enhancers or skin permeation enhancers well known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects.

Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flux can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g., gum acacia or gum tragacanth, naturally occurring phosphatides, e.g. soybean lecithin and sorbitan monooleate derivatives. Examples of antioxidants include, but are not limited to, butylated hydroxy anisole (BHA), ascorbic acid and derivatives thereof, tocopherol and derivatives thereof, and cysteine.

Examples of preservatives include, but are not limited to, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylformamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, Transcutol®, and Azone®.

Examples of chelating agents include, but are not limited to, sodium EDTA, citric acid and phosphoric acid. Examples of gel forming agents include, but are not limited to, Carbopol, cellulose derivatives, bentonite, alginates, gelatin and polyvinylpyrrolidone.

In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane. Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use. Both reservoir and matrix type devices can contain materials such as polydimethylsiloxane, such as Silastic™, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilising agents.

Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches. In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. In embodiments, the adhesive polymer is pa polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer. A matrix dispersion-type system is characterized in that a reservoir of the active ingredient is formed by substantially homogeneously dispersing the active ingredient in a hydrophilic or lipophilic polymer matrix. The drug-containing polymer is then molded into disc with a substantially well-defined surface area and controlled thickness. The adhesive polymer is spread along the circumference to form a strip of adhesive around the disc. A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs. Any of the above-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

### Dosages

When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate symptoms associated with neurodegenerative disease.

In embodiments, the dose of an agent is the maximum that a patient can tolerate and not develop serious or unacceptable side effects.

The dosage range at which teriflunomide exhibits its ability to act therapeutically can vary depending upon its severity, the patient, other underlying disease states that the patient is suffering from, and other medications that may be concurrently administered to the patient. Generally, teriflunomide will exhibit their therapeutic activities at dosages of between about 0.001 mg/kg of patient body weight/day to about 100 mg/kg of patient body weight/day. Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with neurodegenerative disease) is observed in the treated subject, with minimal or acceptable toxic side effects. Applicable methods for determining an appropriate dose and dosing schedule for administration of a pharmaceutical composition of the present invention are described, for example, in Goodman and Goodman et al., Oilman's The Pharmacological Basis of Therapeutics (11th Edition, McGraw-Hill 2005) and Remington's.

Human dosage amounts can initially be determined by extrapolating from the amount of compound used in animals (e.g., mice), as a skilled artisan recognizes it is routine in the art to modify the dosage for humans compared to animal models.

### Combination Therapies

The agents and pharmaceutical compositions described herein can also be administered in combination with another therapeutic molecule. The therapeutic molecule can be any compound used to treat neurodegenerative disease or symptoms thereof. Examples of such compounds include, but are not limited to, an antiglutamergic agent, a neuroprotective agent, an anti-inflammatory agent, an anti-apoptotic agent, a mitochondrial cofactor, an antioxidant, a copper chelating drug, a cyclo-oxygenase inhibitor, and the like.

The agent or pharmaceutical composition can be administered before, during, or after administration of the additional therapeutic agent. In embodiments, the agent described herein for the treatment of neurodegenerative diseases is administered before the first administration of the additional therapeutic agent. In embodiments, the agent described herein for the treatment of neurodegenerative diseases is administered after the first administration of the additional therapeutic agent (e.g., 1, 2, 3, 4, 5, 6, 7, 8, 9, 10, 11, 12, 13, 14 days or more). In embodiments, the agent described herein for the treatment of neurodegenerative diseases is administered simultaneously with the first administration of the additional therapeutic agent.

The amount of therapeutic agent administered to a subject can readily be determined by the attending physician or veterinarian. Generally, an efficacious or effective amount of an agent described herein for the treatment of neurodegenerative diseases and an additional therapeutic is determined by first administering a low dose of one or both active agents and then incrementally increasing the administered dose or dosages until a desired effect is observed (e.g., reduced symptoms associated with neurodegenerative disease), with minimal or no toxic side effects. Applicable methods for determining an appropriate dose and dosing schedule for administration of a combination of the present invention are described, for example, in Goodman and Oilman's and Remington's.

### Kits or Pharmaceutical Systems

The invention provides for kits containing at least one agent described herein for the treatment of neurodegenerative diseases. The kits are suitable for use in preventing or treating neurodegenerative diseases such as ALS. In embodiments, the agent is provided as a pharmaceutical composition. In embodiments, the kit provides instructions for use. The instructions for use can pertain to any of the methods described herein.

Kits according to this aspect of the invention may comprise a carrier means, such as a box, carton, tube or the like, having in close confinement therein one or more container means, such as vials, tubes, ampules, bottles and the like. In embodiments, the kit provides a notice in the form prescribed by a governmental agency regulating the manufacture, use, or sale of pharmaceuticals or biological products, which notice reflects approval by the agency of manufacture, use or sale of the kit and the components therein for human administration.

### Therapy

Therapy may be provided wherever therapy for neurodegenerative disease is performed: at home, the doctor's office, a clinic, a hospital's outpatient department, a hospital, or the like. Treatment generally begins at a hospital so that the doctor can observe the therapy's effects closely and make any adjustments that are needed. The duration of the therapy depends on the kind of disease being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press). Useful techniques for particular embodiments will be discussed in the sections that follow.

### EXAMPLES

It should be appreciated that the invention should not be construed to be limited to the examples that are now described; rather, the invention should be construed to include any and all applications provided herein and all equivalent variations within the skill of the ordinary artisan.

A common neuropathological feature of ALS is the occurrence of a neuroinflammatory reaction consisting of activated glial cells, mainly microglia and astrocytes, and T cells. In SOD1 G93A mice, an increase in T-cell numbers occurs concomitantly with microglial activation, similar to that seen in human beings. Some discrepancy regarding the nature of this increase in T cells exists: one study reported an increase in both CD4+ and CD8+ T cells early in disease, whereas another study detected only CD8+ cytotoxic T cells at end-stage disease. During this process of activation, microglia and astrocytes upregulate expression of a whole subset of cell-surface markers, chemokines and cytokines. Accordingly, W19 (anti-mouse CD52 antibody) and teriflunomide were tested to determine their effect in slowing disease course in ALS mice. W19 and teriflunomide are potent modulators of inflammation and have also been shown to be effective in mouse models of CNS disease (EAE mouse model of multiple sclerosis), which similar to ALS mice exhibit a prominent neuroinflammatory component.

### Example 1

### Evaluation of Dihydroorate Dehydrogenase (DHODH) Inhibitors in ALS Mice

SOD1-G93A transgenic mice (also known as G93A-SOD1 mice) are currently the best available model to test experimental therapeutics for the treatment of ALS. The transgenic mice express a G93A mutant form of human SOD1, and exhibit many of the features associated with human disease. For example, the transgenic mice present with progressive muscular atrophy and weakness. Neuroinflammation, demyelination, and motor neuron death are observed in the mice, and ultimately, the mice become paralyzed and have a shortened lifespan.

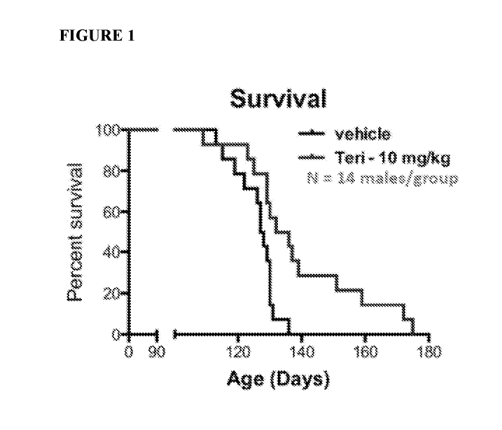

To evaluate the effectiveness of DHODH inhibitors in treating ALS, 10 mg/kg of teriflunomide was administered by oral gavage to 82 day old SOD1-G93A male mice. As shown in FIG. 1, treatment with 10 mg/kg teriflunomide (n=14) significantly increased survival of the SOD1-G93A mice by 7 days as compared to control (n=14).

In the art, therapeutic candidates are generally tested in SOD1-G93A mice prior to symptom onset. In addition, there are no published studies to date (using the study design recommended by the ALS Therapy Development Institute; Scott, S., et al. “Design, Power, and Interpretation of Studies in the Standard Murine Model of ALS.” Amyotrophic Lateral Sclerosis 9.1 (2008): 4-15. SCOPUS. Web. 18 Nov. 2013) that have shown improved SOD1-G93A survival when treatment is initiated after symptom onset. As 82 day old SOD1-G93A mice are clinically symptomatic, these results demonstrate that DHODH inhibitors are effective in treating symptomatic ALS mice.

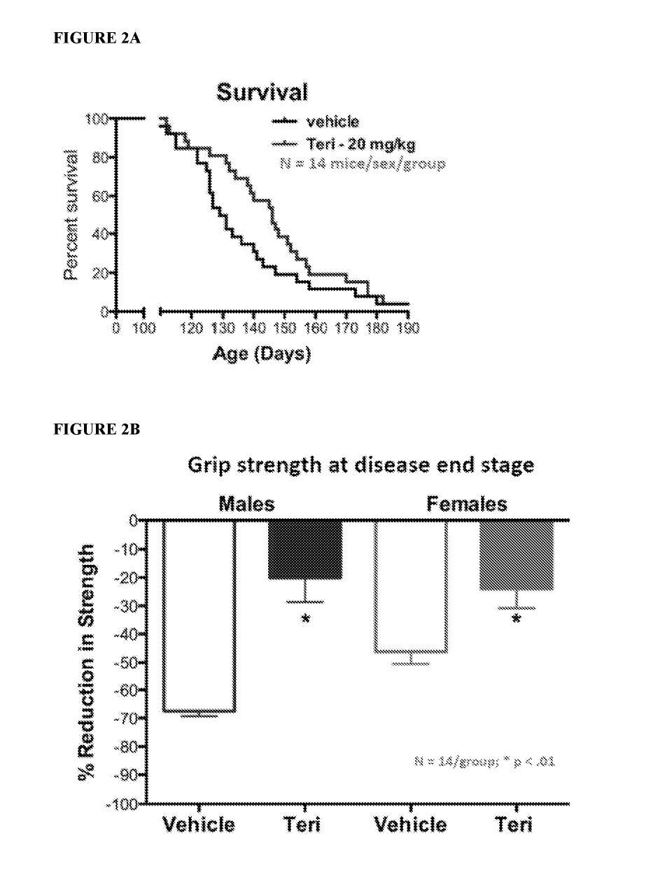

In a second experiment, 20 mg/kg of teriflunomide was administered to 82 day old SOD1-G93A male and female mice (n=14/sex). Survival was assessed, and medial survival of the SOD1-G93A mice was significantly increased by 16.5 days as compared to control (n=14/sex). See FIG. 2A. In addition, treatment with teriflunomide significantly slowed muscle strength loss in SOD1-G93A mice. See FIG. 2B. These results further support the use of DHODH inhibitors in treating ALS.

### Example 2

### Lymphocyte Depletion in ALS Mice

DHODH inhibitors can be used to treat autoimmune disorders such as multiple sclerosis. It is thought that an effect of DHODH inhibition is to limit the expansion of stimulated lymphocytes, thereby reducing the number of available lymphocytes to migrate into the central nervous system. To determine whether the above results were due to precluding lymphocyte entry into the spinal cord, a lymphocyte depletion experiment was conducted using a monoclonal IgG2a mouse anti-mouse CD52 antibody, W19.

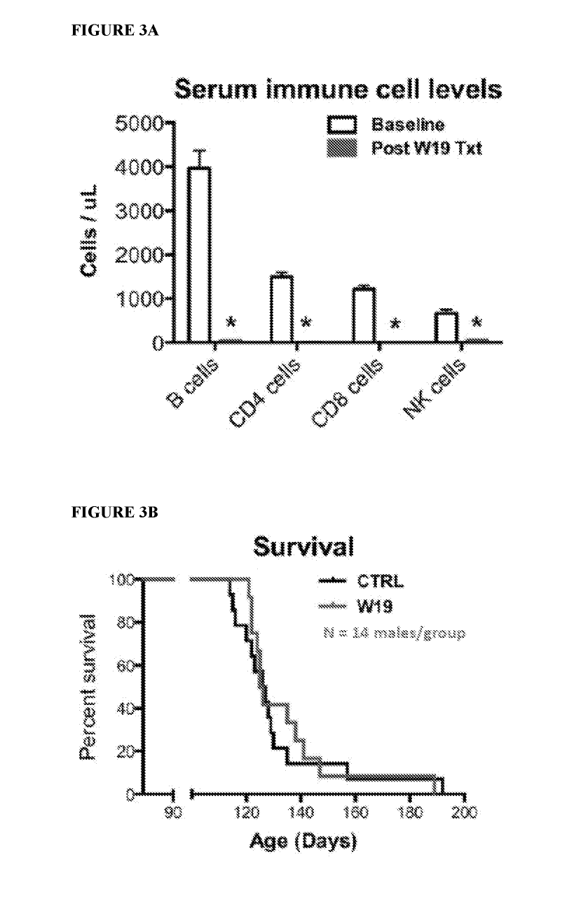

Male SOD1-G93A mice (n=14) were administered W19 (subcutaneous injection at 10 mg/kg for 5 consecutive days and then every 10 mg/kg every 2 weeks thereafter) was administered starting at 82 days of age. After 7 days, peripheral blood was collected and lymphocyte depletion was evaluated using flow cytometry. As shown in FIG. 3A, W19 effectively depleted B cells, CD4⁺ cells, CD8⁺ cells, and NK cells in treated mice. However, unlike teriflunomide, treatment with W19 did not increase survival of SOD1-G93A mice vs. control (n=14). See FIG. 3B. These results indicate that it is unlikely that teriflunomide's efficacy in ALS is mediated only through its ability to block aberrant T cell migration into the spinal cord.

Described herein is a novel method for treating neurodegenerative diseases. As discussed in detail above, it has been discovered that DHODH inhibitors are effective in improving functional and survival outcome in symptomatic ALS mice. Accordingly, the invention features methods for using DHODH inhibitors to treat a subject having or at risk of having a neurodegenerative disease (e.g., ALS). The invention also features compositions for use in treating a subject having or at risk of having a neurodegenerative disease (e.g., ALS).

The results reported herein were obtained using the following methods and materials.

### Animals.

Transgenic male (n=84) and female littermate (n=28) mice that express the mutant SOD1^{G93A} transgene at high-levels were divided to achieve cohort, sibling and sex matching amongst groups for these studies (Scott, S., et al. “Design, Power, and Interpretation of Studies in the Standard Murine Model of ALS.” Amyotrophic Lateral Sclerosis 9.1 (2008): 4-15. SCOPUS. Web. 18 Nov. 2013). Mutant SOD1 gene copy number and SOD1 protein expression were confirmed by PCR and western blot analysis. Animals were housed under light:dark (12:12 h) cycle and provided with food and water ad libitum. All procedures were performed using protocols approved by Genzyme Corporation's Institutional Animal Care and Use Committees.

### Disease Scoring.

Mice were scored into the following phases: presymptomatic (Pre-SYMP)=no visible motor abnormalities (i.e., normal hind limb splay); Symptomatic (SYMP)=abnormal hindlimb splay; End stage (ES)=onset of limb paralysis (typically hindlimb); and Moribund (MB)=unable to right themselves within 30 sec after being placed on their back.

### Grip Strength Analysis.

Testing of motor function using a grip strength device (Columbus Instruments, Columbus, Ohio) began at 60-70 days of age as previously described (Dodge, J. C. et al. (2008) 16:1056-1064). Each weekly session consisted of 4 tests per animal. Data shown represent percent decline in strength from baseline. Baseline measurements were made at 82 days of age. Percent reduction is grip strength is shown for vehicle treated mice scored as ES and age matched sibling mice treated with teriflunomide.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### INCORPORATION BY REFERENCE

All patents and publications mentioned in this specification are herein incorporated by reference to the same extent as if each independent patent and publication was specifically and individually indicated to be incorporated by reference.

What is claimed is:

1. A method of treating amyotrophic lateral sclerosis (ALS) in a subject, wherein the method comprises administering a therapeutically effective amount of a dihydroorate dehydrogenase (DHODH) inhibitor to the subject.

2. The method of claim 1, wherein administration of the DHODH inhibitor slows progression of ALS, reduces intensity of symptoms associated with ALS, delays onset of symptoms associated with ALS, reduces weight loss associated with ALS, reverses weight loss associated with ALS, delays mortality, or combinations thereof.

3. The method of claim 2, wherein the symptoms associated with ALS are selected from group consisting of fine motor function, gross motor function, bulbar function, respiratory function, and combinations thereof.

4. The method of claim 2, wherein the symptoms associated with ALS are selected from the group consisting of muscle twitching, muscle weakness, muscle control, walking, speech, eating, swallowing, writing, climbing stairs, cutting food, turning in bed, salivation, dressing, maintaining hygiene, breathing, dyspnea, orthopnea, respiratory insufficiency, and combinations thereof.

5. The method of claim 4, wherein the subject is at risk of having ALS.

6. The method of claim 4, wherein the subject has been diagnosed with ALS.

7. (canceled)

8. A method for delaying mortality in a subject having or is at risk of having amyotrophic lateral sclerosis (ALS), wherein the method comprises administering a therapeutically effective amount of a dihydroorate dehydrogenase (DHODH) inhibitor to the subject.

9. The method of claim 7, wherein administration of the DHODH inhibitor prevents or delays the onset of respiratory failure.

10. The method of claim 8, wherein the method delays mortality associated with respiratory failure.

11. The method of claim 9, wherein the subject is at risk of having ALS.

12. The method of claim 9, wherein the subject has been diagnosed with ALS.

13. (canceled)

14. The method of claim 11, wherein the ALS is familial ALS.

15. The method of claim 11, wherein the ALS is sporadic ALS.

16. The method of claim 11, wherein the ALS is a mammal.

17. The method of claim 14, wherein the subject is a human patient.

18-20. (canceled)

21. The method of claim 1, wherein the DHODH inhibitor is a small molecule chemical compound, antibody, nucleic acid molecule, polypeptide, or fragment thereof.

22. The method of claim 16, wherein the method is a small molecule chemical compound.

23. The method of claim 17, wherein the DHODH inhibitor inhibits biosynthesis of pyrimidine nucleotides.

24. The method of claim 18, wherein the DHODH inhibitor binds to DHODH.

25. The method of claim 17, wherein the DHODH inhibitor is a substrate-like inhibitor; an isoxazolecarboxanilide or 3-hydroxy-2-cyanocrotanilide; a triazolopyrimidine based inhibitor; a trifluoromethy phenyl butenamide derivative; an ethoxy aromatic amide-based inhibitor; a cyclic aliphatic or aromatic carboxylic acid amide; an aromatic quinoline carboxamide derivative; a 2-phenylquinoline-4-carboxylic acid derivative; an aryl carboxylic acid amide derivative; a cyclopentene dicarboxylic acid amide derivative; a terphenyl carboxylic acid amide derivative; a cyclopropane carbonyl derivative; a biaryl carboxyamide derivative; a biphenyl-4-ylcarbamoyl thiophene/cyclopentene carboxylic acid derivative; an amino-benzoic acid derivative, an N-arylaminomethylene malonate derivative; a 4-hydroxycoumarin, fenamic acid or N-(alkylcarbonyl) anthranilic acid derivative; an alkyl-5-benzimidazole thiophene-2-carboxamide derivative; an amino nicotinic acid or isonicotinic acid derivative; or a salt thereof.

26. The method of claim 20, wherein the DHODH inhibitor is (Z)-2-cyano-3-hydroxy-but-2-enoic acid-(4′-trifluoromethylphenyl)-amide or a salt thereof.

27-31. (canceled)

32. A method for delaying mortality in a human subject having or is at risk of having amyotrophic lateral sclerosis (ALS), wherein the method comprises administering a therapeutically effective amount of a dihydroorate dehydrogenase (DHODH) inhibitor to the subject, and wherein the DHODH inhibitor is (Z)-2-cyano-3-hydroxy-but-2-enoic acid-(4′-trifluoromethylphenyl)-amide or a salt thereof.

33-53. (canceled)
